(19) United States
(12) Patent Application Publication
(10) Pub. No.: US 2016/0251628 A1
(43) Pub. Date: Sep. 1, 2016

(54) Apparatus And Method For Filtration Of A Suspension

(71) Applicants: EXMOOR PHARMA CONCEPTS LTD, Berkshire (GB); LONZA BIOLOGICS PLC, Slough (GB); UCL BUSINESS PLC, London (GB); AMERCARE LTD, Thame (GB)

(72) Inventors: John Vincent, Reading (GB); Rui Tostoes, London (GB); Farlan Veraitch, London (GB); John Dodgson, Slough (GB)

(21) Appl. No.: 15/033,287

(22) PCT Filed: Oct. 30, 2014
(86) PCT No.: PCT/GB2014/053227

§ 371 Date: Apr. 29, 2016

(30) Foreign Application Priority Data
Oct. 30, 2013 (GB) 1319139.0

(51) Int. Cl. C12N 5/071; C12M 1/00; A61K 35/42; B01D 63/08

(52) U.S. Cl. CPC C12N 5/0688; B01D 63/087; C12M 47/02; A61K 35/42; B01D 2315/16; B01D 2313/10; B01D 2313/12

(57) ABSTRACT

The present invention provides devices suitable for filtration including diafiltration of a suspension containing a species such as particles, cells or macromolecules, apparatus comprising such devices and methods of using the devices and apparatus. The devices of the invention comprise a vessel having a filter that divides the vessel into a first chamber and a second chamber provided with various outlet ports. Gas is used to finally concentrate the suspension (118) in the filtration chamber by displacing the liquid up to 95% through the filter (14).

The present invention relates to devices suitable for filtration including diafiltration of a suspension containing a species such as particles, cells or macromolecules, apparatus comprising such devices and methods of using the devices and apparatus.

### BACKGROUND

Prior art devices for filtration and diafiltration of a liquid containing a species in suspension are known to have drawbacks, especially where the aim of the filtration process is to recover the retentate. In the case of a dead-end filter the species accumulates on the filter, restricting flow and resulting in reduced filtration efficiency. In the case that the object is to recover retentate, recovery rates are often low as the retentate adheres to or forms a cake on top of the filter. Tangential flow filtration (TFF, also known as cross-flow filtration), in which the suspension is flowed across a filter such that a majority component of flow in the bulk liquid is parallel to the filter surface, reduces the tendency of the retentate to adhere to the filter and for cake to build up, and is especially suited to provide a continuous flow of filtrate from the filtration process. It may be used to concentrate the retentate, but is essentially a multi-pass process in which a small proportion (typically less than 10%, often less than 5%) of the liquid in the suspension is removed at each pass through the filter, and suffers the disadvantages (i) the species must be pumped around a recirculation pathway through the retentate side of the filter, which is undesirable if the species is easily damaged or tends to adhere to the surfaces of the fluidic pathway; (ii) at higher concentration factors the retentate tends to become denser and more viscous and so harder to recirculate and in some cases, more prone to damage to or agglomeration of the species and (iii) that TFF is usually implemented using a differential pressure across the membrane provided by a pressure drop component in the outlet flow pathway of the retentate side of the filter device, which for easily damaged species may add to the risk of damage, especially at higher concentrations.

Particularly for the case of cell suspensions and processes in which the cells in the retentate are the valued output from the filtration or diafiltration process, such as in cell therapy bioprocessing, prior art devices and apparatus using them are poorly adapted. In particular, small scale TFF systems have a large internal surface area compared with the volume of the sample chamber and the area of the filter, formed by the surfaces of the recirculation system and the pump means, which leads to unacceptable losses of cells by adsorption to the surfaces.

In cell therapy cells having a therapeutic effect are administered to a patient, for example by topical injection or by systemic infusion. The cells may be sourced from the patient themselves in autologous therapy, cultured, selected or otherwise manipulated, sometimes expanded in number, and then transplanted back into the patient. In allogeneic therapies a donor cell line is cultured, cells are treated to add or to enhance efficacy, selected or otherwise manipulated, and then transplanted into a patient. In either case in many production processes the cells need to be cultured in a culture medium that is not suitable for administration to a patient, so which needs to be washed out and replaced by an excipient (a liquid medium usable as a carrier for a therapeutic agent, suitable for use in the human body) before administration. In some cases the volume of the cell suspension needs to be reduced. In some cell therapy production processes a cell suspension may be frozen containing a cryoprotectant for storage and transportation, and needs to be thawed and the cryoprotectant washed out and replaced with an excipient before administration to the patient. Whether the cell suspension is used with freezing or without, there is a need for an apparatus adapted to carry out washing and volume reduction steps and then to enable loading of the product into an administration device ready for use. Such an apparatus needs to be able to process small volumes of valuable cells in suspension, achieving good recovery rates with acceptably low damage to the cells, preferably in a closed system and in an automatic or semi-automatic way. The prior art does not provide apparatus and methods that meet this need.

It is an object of the invention to provide improved filter devices, improved apparatus using filter devices, systems comprising such devices and apparatus for processing suspensions, and methods for filtration of suspensions that overcome these and other drawbacks of the prior art. It will be appreciated that while the devices, apparatus, system and methods will be described herein with reference primarily to cell suspensions, according to the embodiment they are usable for suspensions containing other species, such as particles and solubilised macromolecules.

### PRIOR ART

U.S. Pat. No. 7,713,232, Uber III et al., discloses a device for washing cryoprotectant from a cell suspension comprising a closed-ended vial divided into two portions by a dividing wall, with an inlet at the top of the vial into the first portion, a filter provided in the second portion and an outlet at the top of the vial in the second portion above the filter (see FIG. 10c, 10d). Cells are washed in the base of the vial by liquid flowing from the inlet, downwards through the cells at the base of the vial and out through the outlet, cells being prevented from exiting by the filter. Cells are then unloaded via the inlet at the top of the vial by an upward flow of liquid from the second portion. This device suffers the disadvantage of cells being unloaded by upward flow of liquid, so giving inefficient recovery, and dilution of the cell suspension by the liquid used for unloading. The device is not adapted for volume reduction of the cell suspension.

US2003024877, Amann et al., discloses a cell concentration device comprising a closed ended vertical tube having a filter in a wall of the tube and a flow pathway through the filter to a filtrate chamber, with optional vacuum aspiration via the filtrate chamber to assist flow. In use cell suspension is loaded into the tube and liquid from the suspension flows through the filter, so concentrating the suspension. The tube optionally has a tapered collection region below the filter. Concentrated suspension may then be removed by a pipette or syringe. Also disclosed is a closed tube having in the cap a septum through which the suspension may be loaded and recovered and a breather. This device allows cell concentration but does not allow for efficient washing of the cells: no means is disclosed for efficient mixing of a wash liquid with the cells. The device is for manual operation and is not adapted to be built into automated or semi-automated equipment.

U.S. Pat. No. 6,783,983, Condon et al., discloses a separation device for separating cells from microcarriers in bulk scale bioprocessing. The device has a conical lower compartment having an inlet port at the base of the compartment, a horizontal filter above the lower compartment, and a filtrate chamber and outlet to a collection device. The device is not adapted for washing or for volume reduction of a suspension and the apparatus of which the device is part does not comprise means to flow a wash liquid in through the inlet port or to unload cells through the inlet port.

US2012295352, Antwiler, discloses a cell culture device comprising a vessel having an inverted conical lower portion, a conical upper portion joined to the lower portion at the circumference of the cones, an inlet port at the base of the lower portion and an outlet port at the apex of the upper portion, wherein at least the outlet is partially occluded by a porous material so as to prevent exit of cells through the outlet port. The device is disclosed as part of an apparatus having a fluidic configuration that is not configured to be able for washing or volume reduction of a cell suspension within the device; in particular no means is disclosed to flow a wash liquid upwards through the vessel to a reservoir where the wash liquid is retained; instead liquid flowed through the chamber is recycled through the inlet port.

U.S. Pat. No. 8,394,631, Hampson et al., discloses a cell culture device comprising a fluidic channel having an inlet port and an outlet port, cells being cultured on a surface over which the channels pass. Cells on the surface are washed by passing wash liquid through the channels so as to cause plug flow to displace the culture medium without displacing the cells. No filter means is disposed in the fluidic pathway through the device. The volume of wash liquid within the device may then be reduced by introducing a gas so as to cause a plug flow within the channel and to displace the wash liquid out of the outlet port. This device has the disadvantage that in the absence of a filter, cells are likely to be displaced along the channel and lost. The volume reduction step relies on cells being mobilised off the surface which contradicts the requirement for washing without cell loss. This device is adapted for culture of a high cell density and poorly adapted for washing a sample in which a high cell recovery percentage is desired.

WO97004857, Cui, discloses a filtration apparatus for suspensions using a gas uplift pumping mechanism, comprising a filter device having a vertically oriented filter separating a retentate chamber and a filtrate chamber, the retentate chamber having an inlet port at the base and an outlet port at the top, and a feed suspension reservoir connected to the inlet port by means of a check valve and to the outlet port. Gas under pressure is introduced by means of a T junction into the fluid line from the check valve to the inlet port. Gas/liquid mixture in the first chamber rises, forcing recirculation of the feed suspension from and to the reservoir through the filter device. The feed reservoir is under positive pressure, so forcing filtrate through the filter where it is recovered. The feed suspension is concentrated and may be recovered from the reservoir. This apparatus is again poorly adapted for filtration and volume reduction of a small sample; no collection device is provided to allow complete recovery of the concentrate from the reservoir and no fluidic connection suitable for this purpose is disclosed.

In summary, prior art filtration apparatus of the types described above is generally better adapted for separation of a liquid filtrate from a species such as cells or other particles in suspension, when the filtrate is the desired fraction, than when the retentate cells or particles are the desired fraction. In particular, the prior art devices are poorly adapted for concentration of a species in a small volume of retentate and for the efficient unloading of that small volume from the filter device into a collection device. Where such prior art devices are usable, they are manual devices requiring a complex series of operations by a skilled user and so are not adapted for larger scale or automated use. The prior art apparatus described above each suffer from one or more the following drawbacks: (i) they operate on a large volume scale and are impractical for operation at reduced size; (ii) owing to pressure drop within the system or passing of concentrate through a lengthy or recirculating fluidic pathway they are likely to damage a shear-sensitive retentate particle such as a cell or to suffer losses of cells to walls of the apparatus; (iii) they do not include an effective means for unloading a small volume of concentrated suspension from the filter device; (iv) they do not offer a volume reduction capability within the device; (v) they do not include a filter means to retain the species so rely on slow liquid flow rates and adherent or settled particles, such as settled cells, in low concentration, hence leading to long process times and potential unreliability; (vi) they are inherently manually operated or open system devices not capable of being integrated into an automated or closed-system apparatus.

In particular, the prior art does not address the need for efficient filtration and volume reduction of small volumes of suspension where the retentate particles are the desired fraction, and where the particles may be precious and where high recovery is essential. Specifically in cell bioprocessing where the cell is the desired fraction, as for example in cell production and processing for cell therapy, the prior art apparatus is unsuitable.

### DEFINITIONS

Herein unless otherwise stated a liquid means an aqueous liquid that may comprise solutes, such as water, a salt solution, and acidic or basic solution, a buffer, a physiological liquid such as blood or a blood fraction, seminal fluid saliva or urine, a liquid medium as used in cell culture, cell differentiation, cell processing, transport or cryopreservation, or a liquid medium as used in chemical synthesis, analysis or separation, or in materials processing including minerals extraction. The liquid may comprise a mixture of liquid compounds, such as a mixture of water and miscible organic compounds. Where stated the liquid may be a non-aqueous liquid, comprising one or more liquid components that are non-miscible with water, such as a oil, short chain paraffin or other organic compound in liquid form, which may comprise solutes such as other organic compounds or inorganic compounds soluble in the organic compound.

A suspension is a liquid containing a species in suspension. Herein a suspension is an aqueous suspension unless otherwise stated. The phrase ‘in suspension’ means contained within the liquid and in some cases being hydrated, having surface-associated components from the liquid, having a surface charge resulting from interaction with the liquid, or having a liquid environment around it altered from a bulk environment in the same liquid where the species is not present. In the case that the species is a macromolecule such as a protein, the term suspension herein includes a liquid containing a solubilised macromolecule.

An undesired component may be a solute or a compound mixed with the liquid, a fraction of the liquid or a species in suspension in the liquid alongside a species of interest. For example, an undesired component may be a constituent of a culture medium in a cell suspension, a component of a cryoprotectant solution added to a cell suspension before freezing, such as dimethyl sulphoxide (DMSO), a salt or a stabilisation component in the case that the species is a protein such as an antibody, or a contaminant of the suspension, such as a contaminating solid fraction, protein, endotoxin or virus.

In separating a first species from a second, the first species may comprise particles or other species of a first size or type and the second species particles of a second size or type, for example a first species may be a white blood cell and a second type may be a red blood cell; a first species may be a specific type of cell and a second species may be another type. A first species may be a cell and a second species a non-cell particle. A first species may be an intact cell and a second species may be lysed cell fragments.

‘Single pass filtration’ herein means that the suspension to be filtered is passed through a filter device once. In contrast, TFF as disclosed in the prior art is a multi-pass filtration operation in which the suspension to be filtered is recirculated through the filter device and may pass through it many times during the filtration or volume reduction process.

A species may be one or more of a cell, a vesicle, a liposome, a virus, a microorganism, a bacterium, a particle, for example a solid particle or a semi-liquid particle such as a droplet in an immiscible liquid, for example an aqueous droplet in a non-aqueous liquid or vice versa, a particle formed from a gelled polymer, a nanoscale or microscale particle such as a shell structure, a core-shell particle, a nanotube or a nanorod, an assembly of cells such as a cell cluster or an embryoid body, a microcarrier or other substrate, one or more cells on such a microcarrier or one or more cells bound together by a natural substrate such as a protein or a complex comprising multiple proteins, or a macromolecule such as a nucleic acid, an antibody or another protein. A species may comprise two or more species bound together, for example a cell bound to one or more particles, or two or more particles bound together, for example by a protein-protein or nucleic acid-nucleic acid interaction.

Herein nanoscale means having a largest characteristic dimension of the order of 500 nm or smaller, for example in the range about 500 nm to 5 nm or in the range 100 nm to 10 nm, and in preferred embodiments in the ranges about 500 nm to 50 nm, 100 nm to 10 nm, or 50 nm or smaller. Microscale means having a largest characteristic dimension in the range of the order of 500 μm to 0.1 μm or in the range about 100 μm to 0.5 μm and for example in preferred embodiments in the ranges about 100 μm to 10 μm, about 50 μm to 5 μm, about 10 μm to 1 μm, about 5 μm to 0.5 μm, about 1 μm to 0.1 μm. A shell structure is a substantially hollow particle having a shell that is either closed or nearly so. A core-shell particle is a particle having a core and a shell coating the core, formed from a different material from the core. A nanotube (for example a carbon or boron nitride nanotube) is a hollow elongated nanoscale particle, and a nanorod (for example a gold nanorod) is a solid elongated nanoscale particle, each having a ratio of its largest to its smallest dimension of typically 5 or greater. A microcarrier is a particle adapted for culture of cells adherent to the surface of the particle. Typically microcarriers are microscale particles, for example 125-250 micrometre spheres though they may have other shapes such as flat plates, for example hexagonal in shape (Nunc Microhex). Microcarriers can be made from for example DEAE-dextran (Cytodex, GE Healthcare), glass, polystyrene (SoloHill Engineering), acrylamide, and collagen (Cultispher, Percell). Microcarriers may be porous or non-porous and have a specific surface coating, such as collagen, or surface chemistry, including for example extracellular matrix proteins, recombinant proteins, peptides, and positively or negatively charged molecules.

A particle may be a cell, virus, bacteriophage, plasmid, chromosome, or polymer.

A cell may be a prokaryote cell or a eukaryote cell, or a collection of cells in the form of a tissue or tissue fragment, or other multicellular body, for example an embryoid body. A cell may be for example of the following types: a eukaryote cell such as a plant cell, a plant spore, or an animal cell such as a mammalian cell.

A mammalian cell may be a human cell, for example as listed below:

a keratinizing epithelial cell, such as a keratinocyte, a hair shaft cell, a hair root sheath cell, a hair matrix cell (stem cell), a cell of the wet stratified barrier epithelia such as a surface epithelial cell;
a cell specialized for secretion of hormones such as beta cells of the pancreas, cells of the pituitary gland, or a cell of the gut or respiratory tract;
a cell specialized for metabolism and storage such as a hepatocyte, a liver lipocyte or a fat cell;
an epithelial cell serving primarily a barrier function, lining the lung, gut, exocrine glands, or urogenital tract, an epithelial cell lining closed internal body cavities such as vascular endothelial cells of blood vessels and lymphatics, a synovial cell, a serosal cell, a squamous cell of the ear, a choroid plexus cell (secreting cerebrospinal fluid);
a cell from the following list: squamous cells of the pia-arachnoid, cells of the ciliary epithelium of the eye, corneal “endothelial” cells, a ciliated cell with propulsive function of the respiratory tract, of the oviduct and of the endometrium of the uterus (in a female), of rete testis and ductulus efferens (in a male) or a cell of the central nervous system, a cell specialized for secretion of extracellular matrix, of non-epithelial tissue (connective tissue) such as fibroblasts for example of loose connective tissue, of the cornea, of the tendon, of the reticular tissue of bone marrow, a pericyte of blood capillary, a nucleus pulposus cell of the intervertebral disc, a cementoblast/cementocyte (secreting bonelike cementum of the root of the tooth), an odontoblast/odontocyte (secreting dentine of the tooth), chondrocytes of hyaline cartilage of fibrocartilage of elastic cartilage, an osteoblast/osteocyte, an osteoclast, an osteoprogenitor cell (stem cell of osteoblasts), a hyalocyte of the vitreous body of the eye or stellate cell of the perilymphatic space of the ear;
a contractile cell such as skeletal muscle cells, heart muscle cells, smooth muscle cells, or myoepithelial cells of the iris or of the exocrine glands;
a cell related to blood or the immune system such as a red blood cell, megakaryocyte, macrophages and related cells (monocyte, connective-tissue macrophage, a Langerhans cell (in epidermis), an osteoclast (in bone), a dendritic cell (in lymphoid tissues), a microglial cell (in the central nervous system)), a neutrophil, eosinophil, basophil, mast cell, T lymphocyte (helper T cell, suppressor T cell, killer T cell), B lymphocyte (IgM, IgG, IgA, IgE), or killer cell, or stem cells and committed progenitors for the blood and the immune system;
a cell with sensory and transducing functions such as photoreceptors (rod, cones), hearing, acceleration and gravity, taste, smell, blood pH, touch, temperature, pain and configurations of, and forces in, the musculoskeletal system;
a cell from the following list: an autonomic neuronal cell, a supporting cell of the sense organs and of peripheral neurons such as supporting cells of the organ of Corti, or a supporting cell of the vestibular apparatus, or a supporting cell of the taste bud, or a supporting cell of the olfactory epithelium, or a Schwann cell, or a satellite cell or an enteric glial cell; a neuronal or glial cell of the central nervous system such as neurons or glial cells (astrocytes, oligodendrocytes);
a cell from the following list: a lens cell, a pigment cell such as a melanocyte or a retinal pigmented epithelial cell, a germ cell such as an oogonium/oocyte, a spermatocyte, or a spermatogonium, a nurse cell such as ovarian follicle cell, a Sertoli cell (in the testis), or a thymus epithelial cell, an interstitial cell, or an interstitial cell of the kidney or other organs with pacemaker functions;
a cell from the following list: embryonic stem cells, induced pluripotent stem cells, mesenchymal stem cells, mesenchymal stromal cells, very small embryonic-like stem cells, neuroblasts, myoblasts, hepatoblasts, pancreatic progenitor cells, cardiac progenitor cells, hematopoietic stem cells, retinal progenitor cells, photoreceptor precursor cells, corneal progenitor cells, keratinocyte progenitor cells, adipose stem cells, dental pulp stem cells and all adult cell types originated from embryonic stem cells, induced pluripotent stem cells, very small embryonic-like stem cells and adult stem cells.

A vessel is a container having an interior volume adapted to hold a fluid. A vessel may be substantially rigid or may comprise a flexible wall in the manner of a polymer bag adapted to contain a liquid. A vessel may be formed for example as a void in a solid body; as a void formed by two recesses in the major surface of two components held together to define a substantially closed volume; as a volume defined within a set of solid walls that may have a thickness that differs in different areas of the wall—for example the wall thickness may be small compared with a characteristic dimension of the vessel. A vessel may be formed for example by injection moulding of one or more three dimensional polymer components; by machining, embossing or moulding a number of components that are then bonded or otherwise held together; by extruding a profile, optionally followed by processing the extrusion substantially to close one or both ends. The invention is not limited to a specific size or volumetric capacity of vessel but in certain embodiments is adapted for use with a small vessel, suitable for a small volume of suspension. A vessel herein may have a volume in the range 0.05 ml to 5 l, in the range 0.5 ml to 500 ml, in the range 5 ml to 50 ml, and in preferred embodiments in the range 0.5 ml to 5 ml, in the range 1 ml to 10 ml, in the range 5 ml to 50 ml, in the range 10 ml to 100 ml, in the range 50 ml to 500 ml, in the range 100 ml to 1 l or in the range 500 ml to 5 l. In some embodiments suitable for large volume filtration the vessel may have a volume greater than 5 l.

A filter is an element for separation of the species from a first liquid and from small molecule components dissolved in the first liquid. In some embodiments the filter may allow a first species to pass through it while preventing a second species from passing. The filter may comprise a filter membrane, which in some embodiments is hydrophilic. In embodiments adapted for use with a two phase volume reduction method forming part of the invention the filter is adapted to allow a first liquid from the suspension and where present a wash liquid miscible with the first liquid to pass through it while preventing the second phase from passing through it. In the case that the second phase is a gas, the filter is preferably a hydrophilic membrane having a bubble breakthrough pressure selected to be greater than the maximum pressure applied across the filter in use, such that gas will not break through the filter. In the case that the second phase is a second liquid immiscible with the first the filter is preferably not wetted by the second liquid given prior wetting by the first liquid. According to the embodiment, filter membranes may comprise a material chosen from the non-exclusive group of: hydrophilic membrane materials, regenerated cellulose, Polyvinyl difluoride (PVDF), cellulose acetate, mixed cellulose ester, polyethersulfone, polysulphone or polycarbonate in the case of track-etched membranes such as Nuclepore™ or Cyclopore™ filter membranes (Whatman, UK). In some embodiments the filter membrane may be formed from a hydrophobic filter membrane treated to render it hydrophilic, for example by washing in surfactant. Filters having pore sizes in the range of 0.1 μm to 10 μm are available from a number of suppliers and the devices and methods of the invention are usable with a range of filter types. In some embodiments the filter may comprise a mesh such as a nylon or stainless steel mesh, for example having an opening size in the range 30 to 100 μm as used from filtration of larger particles and cell clusters, agglomerations, sheets or multicell bodies. Filter types used for separating cells from liquid or for removing contaminating microorganisms from cell suspensions typically have a pore size in the range around 0.1 to around 1 μm.

A planar filter is a substantially planar filter component that may be in the form of a substantially flat sheet, or may have deviations from being flat such as corrugations, bowing in one or two dimensions, ripples or dimples. A planar filter comprises a filter curved in one or two orthogonal dimensions, for example a domed filter, having a perimeter substantially in a single plane. The term ‘plane of the filter’ means a plane characterising a planar filter when in situ in a filter device, for example the plane of the majority of the perimeter of the filter or a plane forming the mean position of a corrugated, rippled, dimpled or bowed filter.

An immiscible fluid herein means a fluid that forms a separate phase when in contact with the liquid of the suspension, for example a gas in contact with a liquid, a non-aqueous liquid in contact with an aqueous liquid, and a second aqueous liquid in a two-phase aqueous system as formed for example by solubilised macromolecules provided in one or both phases. In some cases an immiscible liquid may form a mixed phase with the liquid of the suspension. According to the embodiment the filter may be chosen according to the nature of the immiscible fluid to allow the liquid of the suspension to pass through it but not, or to a lesser extent, an immiscible second fluid.

Herein the first chamber refers to the chamber into which the suspension is flowed in use, and corresponds to the retentate chamber in literature descriptions of conventional Tangential Flow Filtration (TFF) devices. The first chamber is not limited to a specific size or volume of but in certain embodiments the invention is adapted for use with a small first chamber volume, for example approximately 10 ml or less, suitable for a small volume of suspension. A first chamber herein may have a volume in the range about 0.05 ml to about 51, in the range 0.5 ml to 500 ml, in the range 5 ml to 50 ml, and in preferred embodiments in the range 0.5 ml to 5 ml, in the range 1 ml to 10 ml, in the range 5 ml to 50 ml, in the range 10 ml to 100 ml, in the range 50 ml to 500 ml, in the range 100 ml to 1 l or in the range 500 ml to 5 l. In some embodiments suitable for large volume filtration the first chamber may have a volume greater than 51. In some embodiments the first chamber may have a first cross-sectional dimension parallel to the filter in the range 5 mm to 500 mm, in the range 10 mm to 200 mm, or in the range 20 mm to 100 mm and in preferred embodiments in the range 5 mm to 50 mm, in the range 10 mm to 100 mm, in the range 50 mm to 500 mm. The first chamber may have a value of the second cross sectional dimension perpendicular to the filter divided by the first cross-sectional dimension parallel to the filter in the range about 0.01 to 2, 0.02 to 0.5, and 0.05 to 0.1.

Herein the second chamber refers to the chamber into which filtrate liquid passes from the first chamber via the filter and corresponds to the filtrate chamber in literature descriptions of conventional TFF devices. The second chamber is not limited to a specific size or volume, and in certain embodiments may have a similar volume to that of the first chamber. In some embodiments the second chamber may have a volume smaller than that of the first chamber, for example serving primarily to collect liquid passing through the filter from the first chamber so that the liquid may be directed to a filtrate reservoir. In some embodiments the second chamber may have a greater volume that the first chamber, for example in order to function as a filtrate reservoir. A second chamber herein may have a volume in the range about 0.05 ml to about 51, in the range 0.5 ml to 500 ml, or in the range 5 ml to 50 ml.

Herein the first region of the first chamber is defined as the region distal to the filter, and more specifically distal to a first end region of the filter membrane. Some embodiments of the filter device have a specific layout of components such that the first region is located in the lower region of the first chamber. Other embodiments without such a specific layout of components may in use be oriented such that the first region is lower than the second.

Herein the second region of the first chamber is defined as the region proximal to the filter relative to the first region, and more specifically proximal to the said first end region of the filter membrane. In some embodiments of the filter device, and in others when in use, the device may be oriented such that the second region lies above the first.

A vertical or horizontal orientation defines the orientation as shown in the figures and as indicated by vertical and horizontal axes.

A port is defined as an opening to a vessel through which a fluid and a species may pass. Where stated a port may be configured such that a fluid may pass through it but a species from the suspension may not, for example where a port comprises a filter in the fluidic pathway through it.

A fluidic pathway means a pathway along which a fluid may flow, such as may be formed for example within tubing or fluidic components such as valves, pumps, manifold and fluidic connection devices, and may be defined at least in part within a solid body component, for example in the form of channels, apertures, junctions, or interfaces between a first body component and a second, and by passages through a porous material such as a filter material, a porous membrane or a venting component such as a hydrophobic porous material adapted to allow gas to pass but not to wet with aqueous liquid, for example porous fluoropolymer (such as VYON™, Porex, UK). A fluidic pathway may comprise a valve means operable to control flow along the pathway, for example to open or to close the fluidic pathway to fluid flow.

The phrase ‘tapers towards a port’ means that at least one cross-sectional dimension of the first chamber decreases as the port is approached. In some embodiments the first chamber tapers in one cross-sectional dimension. In some embodiments the first chamber tapers in two cross-sectional dimensions. Such a taper may comprise a cross sectional shape that remains unchanged, or a shape that changes, as the port is approached. For example the first chamber may have a first shape being one of a rectilinear, triangular, oval, circular, semi-oval or semicircular cross-sectional shape that changes to a second, different shape from the above group as the port is approached. In an embodiment the first chamber tapers from a rectilinear to a circular shape. In some embodiments the port comprises a perimeter in the surface of the first chamber and the cross-sectional shape of the first chamber changes from a first shape at a plane adjacent to the filter to a circular cross-sectional shape, in a direction perpendicular to the axis of the fluidic pathway leading through the port. According to the embodiment a cross-sectional dimension may decrease continuously or discontinuously, for example step-wise. For example the first chamber may comprise a rapid change of cross-sectional dimension at a step or ledge, or at a point of change of shape for example from a square to a circular cross-section. According to the embodiment a taper provides an area of a planar cross-section of the first port at its opening to the first chamber of less than 50%, 50% to 20%, 20% to 5%, less than 5%, or less than 1% of the area of the filter.

A first feature or component being ‘in fluid communication’ with a second means that a fluid may flow from the first to the second or vice versa.

A first feature or component being ‘connected to’ a second herein means the first is ‘in fluid communication with’ or ‘optionally in fluid communication with’ the second. A first component may be connected to a second via a valve. When the valve is open a fluidic pathway is provided between the first component and the second, and when the valve is closed the two are still ‘connected’, i.e. the option of fluid communication is present, but the two are not in fluidic communication and the fluidic pathway no longer exists between them.

‘Directly connected’ herein means that a first component is connected to a second without an intervening component—for example a collection device is directly connected to a filter device if there are no further components such as a valve in the fluidic pathway between them.

A valve means is any means to control flow along a fluidic pathway. A valve means may be a pinch valve, a valve comprising an internal fluidic pathway, and may be controllable to cut off or to limit flow proportionally according the embodiment. A 3-way or 4-way valve means may comprise a plurality of discrete 2-way valves manifolded together or may comprise a multi-position changeover valve. A valve means may be provided as part of a filter device in some embodiments. In other embodiments a valve means may comprise a discrete valve component mounted on, adjacent to or separately from the filter device as part of an apparatus. In some embodiments a valve means may be a check valve. A valve means may also comprise a fluidic actuation means that controls flow along a fluidic pathway. A valve means may comprise a syringe where the plunger is held in place so preventing fluid flow into the syringe, for example held in place by an actuator. A valve means may comprise a pump where flow is limited or prevented when the pump is not actuated.

A valve means may comprise a valve actuator and a flexible tubing portion, wherein the valve actuator is adapted to receive the tubing portion so allowing it to close the fluidic pathway through the tubing portion, so forming a pinch valve. In some embodiments the tubing portion is separable from the valve actuator such that it may be mounted onto the valve actuator and removed from it.

A flow cut-off means is a means to prevent flow along a fluidic pathway. A flow cut-off means has two stable conditions: in a first ‘closed’ condition flow is prevented along the fluidic pathway leading to or through it and in a second ‘open’ condition flow is allowed. A flow cut-off means may be a cut-off valve that has a first open position and a second closed position. A flow cut-off means may also be a device such as a syringe or pump that when stationary prevents fluid flow along a fluidic pathway into the device. A flow cut-off means may comprise means to create backpressure in the fluidic pathway to prevent fluid flow along it, such as a venting pathway comprising a valve, such that when the valve is closed the fluidic pathway does not vent, so causing backpressure preventing a fluid entering the fluid pathway. In some embodiments a flow cut-off means may be a septum such that the fluidic pathway is opened when the septum is pierced. In some embodiments the flow cut-off means may form part of the collection device. In some embodiments the collection device may be a syringe having a plunger whose movement is controlled by an actuator such that while the plunger is held stationary the syringe itself acts as a flow cut-off means to prevent flow along the fluidic pathway into the syringe. In some embodiments the collection device may comprise a pump connected to a container such that when the pump is not actuated the pump acts as a flow cut-off means.

A cut-off valve has a closed condition in which flow along the fluidic pathway through it is prevented and an open condition in which the valve causes substantially no pressure drop along the fluidic pathway at the flow rates intended for use in the apparatus. The invention is not limited to any specific flow rate, and the cut-off valve may be chosen by the skilled person so as not to present a pressure drop through it when open as compared with pressure drops in other parts of the apparatus. In some embodiments a cut-off valve may comprise a pinch valve, a 2-way valve having wetted internal components, or a 3-way or higher-way valve having the capability to close the fluidic pathway between a first port and a second port on the valve.

A check valve is a valve that allows flow in a first direction but not in the reverse direction, and is pressure sensitive: a positive pressure in the direction of allowed flow opens the valve, and a negative pressure in that direction (i.e. a positive pressure in the reverse direction) causes the valve to close.

A source of a fluid may comprise a reservoir containing the fluid such as a rigid reservoir, a collapsible reservoir or bag (a flexible fluid container formed by sealing together of flexible polymer layers), or a syringe, and in some instances may be actuatable in order to control flow of fluid into or out from the reservoir, for example by compressing a bag or applying a head pressure of gas to a liquid within a reservoir. A source of fluid may comprise a gaseous atmosphere external to the apparatus, or a source of fluid from a reservoir connected to the apparatus but remote from it.

A reservoir configured to retain a fluid allows a fluid to flow into it in a first flow direction but does not permit exit of the fluid in the same flow direction. For example, such a reservoir for a liquid may comprise a container having a fluidic inlet and a breather, so allowing liquid to enter the reservoir and gas to leave it, liquid being retained within the reservoir. In some embodiments liquid may exit the reservoir once again if the flow direction is reversed. Such a reservoir may comprise a bag, which inflates as it fills with fluid.

A fluidic actuation means is a means for moving fluid along a fluidic pathway, for example a pump such as a positive displacement pump, a peristaltic pump, a syringe, a source of liquid at a height to provide flow under gravity, pressure in a liquid reservoir such as gas pressure in the headspace above a liquid in a reservoir, and compression of a compressible fluidic reservoir.

A collection device is a device adapted to collect the output of a process carried out in the apparatus, for example a concentrated suspension, and may comprise a passive collection device such as a receptacle or an active collection device comprising a receptacle and a fluidic actuation means. A collection device may comprise one of the following: a container, a vial, a catheter, a tubeset adapted to receive the product before administration to a patient, a flexible bag, a syringe, a syringe with a pre-fitted needle, a pump having a fluidic pathway to a receptacle.

A breather is a device in fluid communication with a reservoir or fluidic pathway adapted to allow gas to pass through it but to prevent passage of liquid out from the reservoir or pathway. For example a breather for use with aqueous liquids may comprise a hydrophobic porous material through which gas may pass but which will not be wetted by an aqueous liquid, or a narrow hydrophobic capillary channel into which an aqueous liquid will not pass. For example a breather may comprise a porous fluoropolymer such as VYON™ as supplied by Porex, UK. A breather for use with non-aqueous liquid may comprise a hydrophilic material that will not be wetted by the non-aqueous liquid.

Washing and diafiltration are taken to have the same meaning herein, that a component of a liquid suspension is reduced in concentration or replaced by one or more components from a second liquid added to the suspension.

A wash liquid refers to a liquid miscible with the liquid of the suspension, adapted to wash a component out from the suspension. Herein the wash liquid and the liquid of suspension are aqueous unless otherwise stated. A wash liquid may be for example water, saline, a buffer, an aqueous solution containing solutes such as salts or sugars for example to control osmolarity, an acid, base or neutral solution. A wash liquid may comprise a complexing agent to complex a component of the suspension. For example, in some embodiments the suspension is a cell suspension comprising a cryoprotectant and the wash liquid is adapted to replace at least a portion of the cryoprotectant. In some embodiments the wash liquid may be an excipient suitable for injection into a patient. In some embodiments the wash liquid has an osmolarity similar to that of the cell suspension to avoid osmotic shock.

A cryoprotectant is a material that when added to a cell suspension allows the suspension to be frozen while allowing the cells to recover on thawing. Examples of cryoprotectants are dimethyl sulphoxide (DMSO) and solutions containing it, typically at between 5 and 10% by volume though in some cases a higher concentration; glycerol, trehalose, sucrose and polyethylene glycol (PEG) in a range of concentrations, and proprietary cryostorage solutions such as Cryostore™ (Biolife, Inc. USA).

An excipient is a liquid medium usable as a carrier for a therapeutic agent, suitable for use in the human body. Examples of excipients include phosphate buffered saline (PBS) or physiological saline solution.

A culture medium is a liquid in which cells may be cultured containing nutrients, buffers, salts and proteins required for cell growth. The composition of the medium may vary depending on the cell type to be cultured.

A closed system is an apparatus whose interior is isolated from the external environment, and within which process steps may be carried out without exposing an interior portion or surface of the apparatus, or its contents or materials that contact the contents during the process, to the external environment, so that following sterilisation of the apparatus it may be used in aseptic processing without needing to be within a clean environment.

### DESCRIPTION OF THE INVENTION

The filter device, apparatus, system and method of the invention is directed to filtration, including diafiltration, of a liquid containing a species in suspension, which is referred to also as a ‘suspension’, for example concentration of the species, washing out an undesired component from the liquid or from within the species, separation of the species from the liquid, or separating a first species within the liquid from a second.

In a particular application and in some embodiments the species are cells and the invention is directed to one or more of separation, filtration, diafiltration, concentration and resuspension of cells. In particular in some embodiments the device apparatus and method are adapted for processing cells in suspension prior to use in cell therapy.

According to a first aspect of the invention there is provided:

a device for filtration of a liquid containing a species in suspension comprising a vessel, the vessel comprising a filter that divides the vessel into a first chamber and a second chamber, the first chamber comprising a first region and a second region wherein the second region is above the first region, the vessel being provided with:
(i) a lower port opening to the first region of the first chamber, the lower port being configured to permit exit of a liquid containing the species from the first chamber,
(ii) an upper port opening to the second region of the first chamber, and
(iii) an outlet port opening to the second chamber.

In some embodiments the device is provided with a single lower port opening to the first region, the lower port being configured also to receive a liquid containing the species. In some embodiments the device is provided with two or more lower ports opening to the first region, a first lower port being configured to permit exit of a liquid containing the species and a second lower port being configured to receive a liquid containing the species.

In some embodiments the device is provided with an upper port configured to receive a liquid containing the species.

According to a second aspect the invention provides an apparatus for filtration of a liquid containing a species in suspension comprising:

a filter device comprising a vessel, the vessel comprising a filter that divides the vessel into a first chamber and a second chamber, the first chamber comprising a first region distal to a first end of the filter and a second region proximal to the said first end of the filter, the vessel being provided with:
(i) a first port opening to the first region of the first chamber, the first port being configured to receive a liquid containing the species and further configured to permit exit of the liquid containing the species from the first chamber, and
(ii) an outlet port opening to the second chamber,
and
a fluidic system in fluid communication with the filter device comprising:
(i) a collection device configured to receive a liquid containing the species from the first port
(ii) a first reservoir configured to contain a second fluid and a first fluidic pathway from the first reservoir to the first chamber,
(iii) a second reservoir configured to retain liquid from the filter device and a second fluidic pathway from the second chamber to the second reservoir, and
(iv) a fluidic actuation means provided in one of the first or the second fluidic pathways.

In this way the apparatus is configured to move the second fluid from the first reservoir to the first chamber and configured to permit fluid to flow from the second chamber to be retained in the second reservoir.

In some embodiments the apparatus comprises a filter device wherein a second port is provided opening to the second region of the first chamber.

In some embodiments the apparatus comprises a filter device wherein the second port is provided in the form of a breather permeable to gas but impermeable to the suspension.

In some embodiments the breather comprises a hydrophobic porous material adapted to allow passage of gas but not of aqueous liquid, such as a porous fluoropolymer.

In some embodiments the second region of the first chamber is above the first region, the first port being a lower port and the second port being an upper port.

In some embodiments the filter device is provided with a lower port that opens at or adjacent to the lowest point in the surface of the first chamber.

In some embodiments the first region tapers towards the lower port as defined herein. In some embodiments provided with two or more lower ports, the first region tapers towards a lower port configured to permit exit of a liquid containing the species.

In this way the filter device and an apparatus are adapted to achieve filtration or diafiltration of a liquid containing a species in suspension introduced to the first chamber, by receiving the liquid into the first chamber via one of a lower or (where present) an upper port in the first chamber, flowing a further liquid into the first chamber to cause diafiltration or washing of the suspension, and flowing the filtrate to a waste container, the device then being configured to permit exit of the filtered or washed suspension through a lower port in the lower region of the first chamber, in some embodiments at the lowest point in the first chamber, so permitting efficient draining of the suspension into a collection device in fluidic communication with the lower port.

In some embodiments a port is an opening in the surface of the first chamber. In some embodiments a port is an opening to a conduit extending through a surface of the first chamber. Such a conduit may comprise a fluidic pathway defined within a tubular feature extending through a surface of the first chamber, for example formed by a tubular component mounted through the surface.

In some embodiments the vessel has a cross-section that is rectilinear, triangular, oval or circular. In some embodiments the first chamber has a cross-section that is rectilinear, triangular, oval, circular, or semicircular.

In some embodiments the first chamber comprises a lower wall and a planar upper wall spaced apart from the lower wall, wherein the filter forms part of the upper wall.

In some embodiments at least a region of a wall of the first chamber is inclined at a first angle to the plane of the filter, the plane of the filter being as defined herein.

In some embodiments the first angle is between approximately 3 and approximately 30 degrees.

In some embodiments the first angle is between approximately 30 and approximately 85 degrees.

In some embodiments comprising an upper port opening to the upper region of the first chamber, the first chamber tapers towards the upper port.

In some embodiments the device and apparatus of the invention are configured for filtration of a liquid containing cells in suspension.

In some embodiments the invention provides an apparatus comprising a filter device as described above and further comprising a collection device connected to the lower port, the apparatus being configured to allow flow of a suspension from the lower port to the collection device.

In this way such embodiments are configured to permit exit of the suspension to a collection device from the first chamber via a port within the lower region of the first chamber, so allowing ready draining of the chamber with assistance from gravity.

In some embodiments an apparatus comprising a filter device as described above further comprises a reservoir containing a second fluid in fluid communication with the first chamber.

In some embodiments the second fluid is a liquid. In this way in some embodiments the apparatus is configured for diafiltration of the suspension, the second fluid being a wash liquid chosen to wash out an undesirable component of the liquid in the suspension.

In some embodiments the second fluid is a gas. As will be described with regards to a method according to the invention and referring to embodiments of the invention below, in this way a gas may be used to replace liquid within the first chamber, the liquid flowing through the filter to the second chamber, so as to effect volume reduction of the suspension within the first chamber.

In some embodiments the second fluid may be a liquid immiscible with the liquid in the suspension, and the filter is adapted to permit passage of the liquid of the suspension but to prevent passage of the second immiscible liquid. In this way the apparatus is configured for volume reduction of the liquid phase of the suspension, the immiscible liquid acting to replace the liquid of the suspension within the first chamber. The concentrated suspension may then exit the first chamber through the lower port. In the case that the second immiscible liquid is less dense than the liquid of the suspension, the second liquid will remain as a separate phase above the concentrated suspension, allowing the latter to be withdrawn from underneath the second liquid for example by being drawn into a collection device connected to the first port.

In some embodiments the second fluid is an aqueous liquid immiscible with the aqueous liquid of the suspension, for example containing a solubilised macromolecular component that renders it immiscible with the liquid of the suspension. In some embodiments the suspension also comprises a solubilised macromolecular component that acts to render the liquid of the suspension immiscible with the second fluid. In some embodiments the first and second immiscible aqueous liquids are chosen such that a species partitions into the denser of the two liquids. In this way the more dense liquid may be allowed to separate in the lower region of the first chamber while the less dense upper liquid may pass through the filter. The denser fraction comprising the species may then be unloaded from the first chamber to a collection device.

In some embodiments the device is provided with an upper port configured to receive a second fluid immiscible with the liquid of the suspension. In some embodiments the upper port is configured to receive a gas.

In some embodiments the filter is inclined at a second angle to the horizontal. For example the second angle may be in the range approximately 45 to approximately 90 degrees.

In this way the filter may be provided above the first, lower region of the first chamber such that a species tending to sediment within the suspension will tend to sediment away from the filter. In some embodiments the filter is provided substantially vertical.

In some embodiments the filter device comprises a housing having a filter permanently bonded to the housing. In some embodiments the filter device comprises a filter that is removable from the vessel. In some embodiments the filter device is adapted to be disassembled to allow the filter to be replaced. In some embodiments the filter is provided in a holder adapted to interfit with the housing to form the vessel and the first and the second chambers.

In some embodiments an apparatus according to the invention further comprises a control means configured to control movement of fluids into and out from the filter device. In some embodiments the control means comprises one or more power supply switches to control the power supply to the pumps and actuators. In some embodiments one or more such switches are manually-operated switches. A control means may comprise a computer controlled electronic system that interprets instructions in the form of data and provides an output to control one or more actuators configured to control movement of fluids into and out from the filter device. The actuators may be configured to control movement of fluids in one or more fluidic components or pathways connected to the device, for example in a fluidic unit as described below. The control means may comprise a computer such as a microprocessor, a data storage means readable by the computer and a user interface. The control means may comprise communication means to communicate data to or from a display or a further electronic system or data store. The apparatus further comprises a power supply to power the control means and optionally actuation means controlled by the control means.

The control means may be configured to receive data entered by a user, received from a remote electronic system or data source, read from a data storage means associated with the filter device such as with a fluidic unit as described below, and to control movement of fluids in response to such data. The control means may be configured to output data to a display, a printer, a remote electronic system, computer or data storage means, such as a data storage means associated with a fluidic unit comprising the filter device.

In some embodiments the filter device and apparatus comprising it are adapted to form a closed system for processing a suspension. Such embodiments may be adapted for aseptic processing, for example for processing a cell suspension in bioprocessing, such as in processing a cell suspension for cell therapy.

Accordingly, in some embodiments the invention provides a fluidic unit comprising a filter device as described herein and one or more fluidic pathways provided between the filter device and components of the fluidic unit. The fluidic unit may comprise a reservoir connected to the filter device.

In some embodiments the invention provides an apparatus comprising a fluidic unit and a processor, together configured to carry out a process within the fluidic unit, the fluidic unit comprising the filter device and being adapted to interfit with the processor, the processor comprising one or more actuators configured to control fluid flow within the fluidic unit and a control means configured to control the actuators. In some embodiments the actuators are valve actuators.

In some embodiments the fluidic unit comprises:

a filter device as described herein,
an inlet adapted to receive a suspension,
a port opening to a first chamber of the filter device,
a fluidic pathway connecting the inlet to the port,
a fluidic pathway configured to introduce a second liquid to the first chamber, and
an outlet fluidic pathway leading from a port opening to the first chamber.

In some embodiments the fluidic unit comprises a collection device detachably connected to the outlet fluidic pathway.

In some embodiments the fluidic unit further comprises one or more of: a reservoir containing the second liquid connected to the first chamber of the filter device; a waste reservoir connected to the second chamber; a reservoir of gas connected to the first chamber.

In some embodiments the fluidic unit is configured to provide a closed system within the filter device, other fluidic components and fluidic pathways connected to them. In some embodiments the fluidic unit is formed from materials capable of being sterilised as known in the art, such that the closed system is adapted for use in aseptic processing of a cell suspension.

In some embodiments the fluidic unit comprises a flow cut-off means connected to a port opening to the first chamber.

In some embodiments the flow cut-off means comprises a syringe forming part of the fluidic unit. In some embodiments the flow cut-off means comprises a septum. In some embodiments the flow cut-off means comprises a cut-off valve comprising a fluidic pathway within the fluidic unit and an external valve actuator configured to close the said fluidic pathway.

In this way in some embodiments the filter device and apparatus are adapted to achieve processing of a cell suspension, for example for use in cell therapy, in which a component is at least partially removed from the cell suspension by the washing process, and may be replaced by the wash liquid. The wash liquid may be an excipient, and thereby the suspension is processed ready for administration to a patient.

The fluidic unit may comprise one or more reservoirs in the form of a bag, a vial or a syringe, and one or more connections between the filter device and a reservoir may be formed using tubing, for example flexible tubing, as known in the art.

The fluidic unit may comprise additional components as described herein, for example one or more of the following: further inlets, for example for a second liquid such as a wash liquid and a fluid immiscible with the suspension for use in volume reduction; one or more sensors such as a flow sensor or a pressure sensor; one or more breathers adapted to allow passage of gas into or out from a fluidic pathway or reservoir while preventing the entry of pathogens into the fluidic pathway or reservoir. The fluidic unit may comprise one or more reservoirs containing fluids, for example a reservoir containing a wash liquid or a reservoir containing an immiscible fluid for use in volume reduction, such as a gas.

The fluidic unit may comprise a tube set comprising a filter device connected to one or more reservoirs in the form of a bag or a syringe by means of flexible tubing as known in the art.

The fluidic unit may comprise a tube set as defined above and a housing adapted to contain the tube set and to retain the components of the tube set in a chosen configuration. In this way the fluidic unit may be mounted on the processor such that the tube set is located by the housing such that the processor may control fluid flow within the fluidic unit. It will be understood that the housing may retain the tube set while allowing a range of movement of one or more components or regions of the tube set so as to allow an actuator provided on the processor to come into contact with a portion of the tube set.

The processor may comprise one or more actuators such as valve actuators, configured to come into contact with and to move a region of the fluidic unit, for example to move a syringe plunger, to compress a bag, to compress a region of flexible tubing to move fluid within the tubing or to close the fluidic pathway through the tubing.

The apparatus may comprise one or more sensors for properties of a fluid, such as for flow, pressure, presence of liquid or gas (such as bubble sensors), a chemical sensor such as a pH sensor or dissolved oxygen sensor, a conductivity or turbidity sensor; a level sensor to detect the level of a liquid in a reservoir; a temperature sensor, for example to measure temperature of a fluid within a fluidic pathway, of a component of the fluidic unit such as the filter device, or of a region within the fluidic unit; a proximity sensor or an orientation sensor, as may be used for example to detect correct mounting of the unit on a processor, or connection of a fluidic component such as a collection device to the fluidic unit. Such sensors may comprise a reading portion forming part of the processor and a fluidic sensor portion forming part of the fluidic unit and comprising a fluidic pathway, together forming a sensor, the reading unit providing data to a control means forming part of the processor.

The fluidic unit may comprise a data storage means that may be read by a data reader provided as part of the processor. Such a data storage means may comprise one or more of a bar code, a 2D bar-code, a printed label, a magnetic data storage means such as a magnetic strip, an electronic data storage means such as a memory chip, a passive (read only) RFID chip or an active (read/write) memory chip. The processor may comprise means to read a data storage means associated with the fluidic unit.

The fluidic unit may comprise one or more electronic components such as sensors, indicators, switches or data storage devices configured to interact with one or more electronic components provided as part of the processor, and electrical contact means to make contact with contact means provided on the processor.

In some embodiments the apparatus comprises temperature control means to control the temperature of the filter device and optionally of further fluidic components connected to it. In some embodiments comprising a fluidic unit as described herein the apparatus comprises temperature control means to control the temperature of part or substantially the whole of the fluidic unit. A temperature control means may comprise a heater, a temperature sensor and a temperature control device adapted to control the heater in response to data from the temperature sensor. In some embodiments the temperature sensor is positioned to measure the temperature of a region of the filter device. In some embodiments comprising a fluidic unit and a processor comprising a control means, the control means is configured to receive temperature data from the temperature sensor and to control the operation of the heater. In this way the apparatus is configured to achieve processing of a suspension, for example a cell suspension, at a controlled temperature.

According to a further aspect the invention provides a system comprising an apparatus as described herein comprising a filtration device, a control means configured to control movement of fluids into and out from the filter device and a data source in data communication with the control means, wherein the data comprises instructions to determine the movement of fluids in the apparatus.

In some embodiments the data source comprises a computer and a database external to the apparatus.

In some embodiments the data source comprises a data store physically associated with a reservoir housing the liquid suspension to be introduced to the filter device.

In some embodiments the control means comprises means to monitor one or more parameters associated with the process, such as temperature, and to record the parameters in a data storage means. In some embodiments the control means is configured to provide data resulting from the process in a report or to communicate such data to a remote computer system.

In some embodiments a system according to the invention is configured for processing a liquid comprising cells, the system further comprising a source of liquid containing cells in fluid communication with a port leading into the first chamber.

According to a further aspect the invention provides a method for filtration of a suspension using a filter device or an apparatus as described herein, the method comprising the steps of:

(i) introducing the suspension into the first chamber,
(ii) introducing a second fluid into the first chamber, and
(iii) unloading the suspension from the first chamber through a port opening to the first chamber.

In some embodiments of the method the second fluid is a liquid. In some embodiments the second fluid is a wash liquid or excipient.

In some embodiments the second liquid is introduced through a lower port opening to the first region of the first chamber.

In some embodiments the second liquid is introduced through both of a lower port opening to the first region of the first chamber and an upper port opening to the second region.

In some embodiments the second fluid is a gas. The gas may be for example an inert gas, nitrogen, air, artificial air, air comprising CO₂ or an N₂/O₂/CO₂ mixture, to enable pH balancing with a suspension containing a bicarbonate buffer.

In some embodiments the method comprises the steps of introducing a second liquid into the first chamber and then introducing a gas into the first chamber.

In some embodiments the gas is introduced through the upper port opening to the second region of the first chamber.

In some embodiments the gas is introduced into the first chamber until the volume of liquid within the first chamber is reduced by between approximately 50% and approximately 98%, and in some embodiments until the volume of liquid within the first chamber is reduced by between approximately 60% and approximately 95%.

In some embodiments the gas is introduced into the first chamber by application at a port opening to the first chamber of a pressure greater than atmospheric pressure. Atmospheric pressure may be taken to be a mean of 15 psi at sea level. For example the pressure may be in the range atmospheric pressure plus up to 15 psi, atmospheric pressure plus 0.5 to 1.5 psi, atmospheric pressure plus 1 to 3 psi, atmospheric pressure plus 2 to 5 psi, atmospheric pressure plus 2.5 to 15 psi, or atmopheric pressure plus 5 to 50 psi.

In some embodiments the gas is introduced into the first chamber by application of a pressure at the outlet port of the second chamber less than atmospheric pressure. For example the pressure may be in the range atmospheric pressure minus up to 1 psi, atmospheric pressure minus 0.5 to 1.5 psi, atmospheric pressure minus 1 to 3 psi, atmospheric pressure minus 2 to 5 psi, atmospheric pressure minus 2.5 psi to vacuum.

In some embodiments the method comprises the steps of:

(i) introducing a suspension into the first chamber.
(ii) preventing flow through the outlet fluidic pathway from the first chamber.
(iii) introducing a wash liquid into the first chamber.
(iv) introducing a gas into the first chamber to displace liquid through the filter and effect volume reduction in the first chamber.
(v) permitting flow through the outlet fluidic pathway from a first port opening to the first chamber and unloading the liquid containing the species from the first chamber.

In some embodiments the method comprises the step of first priming the device with liquid.

In some embodiments steps (iii) and (iv) above are repeated in order to achieve diafiltration by means of sequential volume reduction and dilution.

In some embodiments the method comprises a step of allowing a species in the sample to sediment within the first chamber.

In some embodiments the method comprises a backflush step of introducing liquid from the second chamber into the first chamber through the filter.

In some embodiments the method comprises the step of tilting the device during the filtration process from a first position at which the lower wall of the first chamber is at a first angle to horizontal to a second position at which the lower wall of the first chamber is at a second angle to horizontal.

In some embodiments the device is vibrated during at least a portion of the filtration process.

In some embodiments the method includes a step wherein the control means reads instructions from a data source so as to control the flow of at least one fluid into or out of the filter device.

In some embodiments the suspension is a cell suspension comprising a cryoprotectant.

In some embodiments the method comprises a step of resuspending the cell suspension in a pharmaceutically acceptable diluent.

A method of use of the embodiment of an apparatus comprising a fluidic unit and a processor comprises the following steps:

(i) the fluidic unit comprising a filter device as described herein is mounted on a processor,
(ii) a source of suspension is connected to the inlet,
(iii) the control means is initiated to follow a program to carry out a filtration process within the fluidic unit under the control of a control means, and
(iv) processed suspension is unloaded from the filter device to a collection device.

In some embodiments the method further comprises the step of connecting a collection device to an outlet of the fluidic unit.

In some embodiments the method further comprises the step of disconnecting a collection device from an outlet of the filter device.

According to a further aspect the invention provides a cell suspension obtained by a method as described herein.

Preferred features of the second and subsequent aspects of the invention are as for the first aspect mutatis mutandis.

### BRIEF DESCRIPTION OF THE FIGURES

FIG. 1 shows a first embodiment of a device according to the invention together with example external fluidic components.

FIG. 2 shows a first embodiment of a device according to the invention together with further example external fluidic components.

FIG. 3 shows a first embodiment of a device according to the invention together with further example external fluidic components.

FIG. 4 shows a second embodiment of a device according to the invention together with example external fluidic components.

FIG. 5 shows a further embodiment of a device according to the invention together with further example external fluidic components.

FIG. 6a shows a further embodiment of a device according to the invention with a suspension within the first chamber.

FIG. 6b shows the embodiment of FIG. 6a with a gas/liquid meniscus at a first position within the first chamber.

FIG. 6c shows the embodiment of FIG. 6a with a gas/liquid meniscus at a second position within the first chamber.

FIG. 7 shows a further embodiment of a device according to the invention.

FIG. 8 shows the embodiment of FIG. 7 positioned at an angle to horizontal.

FIG. 9a shows a cross sectional view of a further embodiment of a device according to the invention.

FIG. 9b shows a plan view of the first chamber of the embodiment in FIG. 9a at the level of the filter membrane.

FIG. 10 shows a further embodiment of a device according to the invention.

FIG. 11 shows the embodiment of FIG. 9a together with example external fluidic components.

FIG. 12 shows a further embodiment of a device according to the invention together with example external fluidic components.

FIG. 13 shows a further embodiment of a device according to the invention together with example external fluidic components.

FIG. 14 shows a further embodiment of a device according to the invention together with example external fluidic components.

FIG. 15 shows a vertical cross-sectional view of a further embodiment of a device according to the invention together with example external fluidic components.

FIG. 16 shows a horizontal cross-sectional view of a first variant of the embodiment shown in FIG. 15.

FIG. 17 shows a horizontal cross-sectional view of a second variant of the embodiment shown in FIG. 15.

FIG. 18 shows a flow diagram showing steps in a method according to the invention.

FIG. 19 shows an isometric view of an embodiment of a device as shown diagrammatically in FIGS. 9 and 10 and two components making up the device.

FIG. 20 shows an isometric view of an embodiment of a device as shown diagrammatically in FIGS. 9 and 10 and two components making up the device, this device being used in examples as described herein.

FIG. 21 shows a diagram of the dimensional parameters of embodiments as shown in FIGS. 19 and 20 with example values of the parameters.

FIG. 22 shows an embodiment of a fluidic unit configured for use with a processor, comprising a filter device according to the invention.

### DETAILED DESCRIPTION OF THE EMBODIMENTS

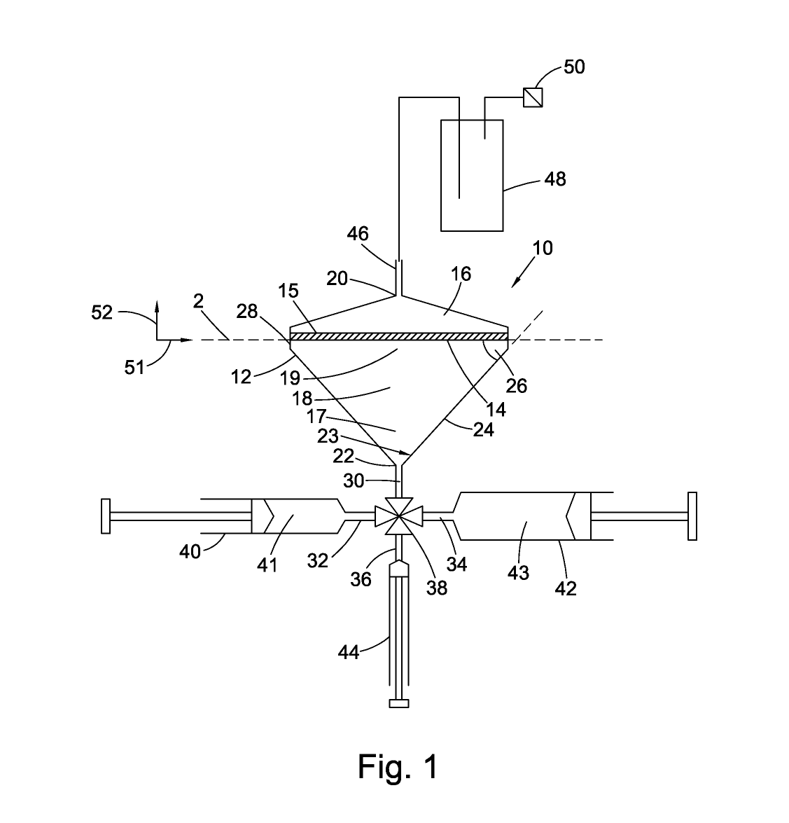

Referring to FIG. 1, a first embodiment of an apparatus according to the invention comprises a filter device 10 comprising a vessel 12 and a filter 14 that divides the vessel into a first chamber 18 and a second chamber 16, the first chamber comprising a first region 17 distal to a first end 15 of the filter and a second region 19 proximal to the said first end 15, the vessel being provided with a first port 22 opening to the first region 17 of the first chamber, the first port being configured to receive a liquid containing the species and to permit exit of the liquid containing the species from the first chamber; and an outlet port 20 opening to the second chamber, and a fluidic system in fluid communication with the filter device 10 comprising:

(i) a collection device 44 configured to receive a liquid containing the species from the first port 22, by means of a fluidic pathway 30 leading from the port 22, a collection fluidic pathway 36 to the collection device 44, and valve means 38;
(ii) a first reservoir 42 configured to contain a second fluid 43, here in the form of a syringe, and a first fluidic pathway 34, 30 from the first reservoir to the first chamber,
(iii) a second reservoir 48 configured to retain liquid from the filter device 10 and a second fluidic pathway 46 from the second chamber 16 to the second reservoir 48, and
(iv) a fluidic actuation means provided in the first fluidic pathway, here comprising the syringe 42.

In this embodiment the filter device 10 is preferably oriented as shown in FIG. 1, such that the second region 19 proximal to the filter lies above the first region 17, with an axis 2 of the device substantially horizontal as shown with reference to horizontal and vertical axes 51 and 52. Axis 2 is parallel to a plane of the filter as defined herein. It will be understood that the invention is not limited to the axis 2 being horizontal in use, and in some embodiments axis 2 may be inclined at an angle to horizontal. In the following, the location and function of the components of the apparatus will be referred to in the orientation as shown in FIG. 1 with the first region 17 of the first chamber referred to as the lower region and the second region 19 as the upper region.

In this embodiment the first port 22 is configured to receive a liquid 41 containing the species and further configured to permit exit of the liquid containing the species from the first chamber (i) by virtue of its position below the filter 14 and distal from it, so that in use the species in the liquid tend to sediment away from the filter, so reducing the tendency common in prior art devices to clog the filter during a filtration process, (ii) in that preferably the first port 22 is located at or adjacent to the lowest point 23 in the first region 17 of the first chamber to permit unloading of a liquid containing the species from the port assisted by gravity, and (iii) the first region 17 of the first chamber 18 tapers towards the first port, so allowing ready draining of the liquid containing the species in use.

The first chamber 18 tapers towards the first port 22. The first chamber has a tapering form wherein a horizontal cross-sectional dimension decreases within at least the lower region 17 towards the bottom of the first chamber, and the first port 22 is located at or adjacent to the lowest point 23 of the chamber. The vessel is defined by a wall 24, at least one portion of the wall within at least the lower region 17 sloping towards the lowest point 23 of the chamber. The said wall portion slopes at an angle 26 to the axis 2, which according to the embodiment is in a range between approximately 3 and 80 degrees, and in some embodiments in the range 30 to 60 degrees. The first chamber may have an inverted frustoconical shape in at least the lower portion 17 of the chamber, with the first port 22 at or adjacent to the apex of the truncated cone. The second chamber 16 preferably also tapers towards the outlet port 20, which is preferably provided at or adjacent to the highest point in the second chamber, and may have a frustoconical shape. The first chamber may optionally have a substantially vertical portion 28 below the filter and above the tapering lower portion.

The filter device 10 may be formed as a thin walled housing defining the vessel 12 as shown in FIG. 1 or in some embodiments may be formed within a solid body, in which the external profile of the body may be independent of the shape of the internal wall 24. The filter may be permanently bonded to the housing or in some embodiments the housing may be adapted for disassembly to allow the filter to be replaced.

The filter device 10 provides a means to introduce a suspension 41 comprising a species in a first liquid into the first chamber by means of first port 22, and then to flow the second fluid 43, in this embodiment being a wash liquid, into the first chamber via the same port so as to wash the suspension. The first liquid and wash liquid then flow through the filter 14 to the outlet port 20, while the species is retained in the first chamber by the filter. In the case that the species tends to sediment, for example if the species comprises cells or particulates, the species will tend to settle into the tapering lower portion 17 of the first chamber. In use upward flow of the wash liquid from the first port 22 into the chamber 18 tends to wash the species upwards into the outwardly tapering space, in which the upward velocity of the inflowing wash liquid is reduced, reducing the upwards force on the species, which tend to settle once again. In some embodiments a recirculating flow of the species is thereby created, resulting in efficient washing. The tapering shape and the height of the filter 14 above the first port 22 result in the species spending less time close to the filter than in other arrangements, so reducing the tendency of the filter to clog.

FIG. 1 shows the filter device 10 as part of an apparatus comprising an external fluidic system configured to allow introduction of the suspension to the filter device and washing as described above. The first port 22 is connected to an inlet fluidic pathway 30, in turn connected to a first 32, a second 34 and a third 36 fluidic pathways, optionally selectable by means of a valve means 38. The valve means may comprise a 4-way valve as shown, or a functionally equivalent arrangement of 2-way or 3-way valves. A source 40 of suspension 41 to be filtered is connected to the first fluidic pathway 32, a source 42 of wash liquid is connected to the second fluidic pathway 34 and a collection device 44 for the filtered suspension is connected to the third fluidic pathway 36. In the system of FIG. 1 the sources 40, 42 are shown as filled syringes and the collection device 44 as an empty syringe that may be actuated by actuators (not shown), but in some embodiments of the apparatus one or more sources may instead comprise reservoirs and fluidic actuation may be by other means as defined herein, for example pumps, gravity, gas pressure in the headspace above a liquid in a rigid fluidic reservoir or compression of a compressible fluidic reservoir. In some embodiments, for example where the fluidic actuation means is self-sealing against backpressure, as for example syringes coupled to actuators, the valve means 38 may be replaced by a direct fluidic connection between two or more fluidic pathways, backflow into a non-flowing fluidic pathway from a flowing pathway being prevented by backpressure. In some embodiments the fluidic pathway 30 is connected to the third fluidic pathway 36 to the collection device directly without passing through a valve. This allows unloading of the washed suspension while reducing the possibility of the suspension being partially held up or lost within the valve. The outlet port 20 from the second chamber is connected to an outlet fluidic pathway 46, connected in turn to a second, or waste, reservoir 48, optionally vented by a breather 50.

Fluidic pathways may be provided for example within tubing, fluidic channels within a body structure, or fluidic manifold components assembled together as known in the art. The filter vessel and one or more fluidic pathways may be formed within a common device, for example formed from moulded or machined polymer. Fluidic connection components and further tubing may be used to connect external components such as the reservoirs and fluid actuation means to the device. One or more reservoirs 40, 42, 48 may be provided as part of a common device together with the vessel 12 and fluid flow into or out from the reservoirs may be actuated by actuators external to the common device by means as described above.

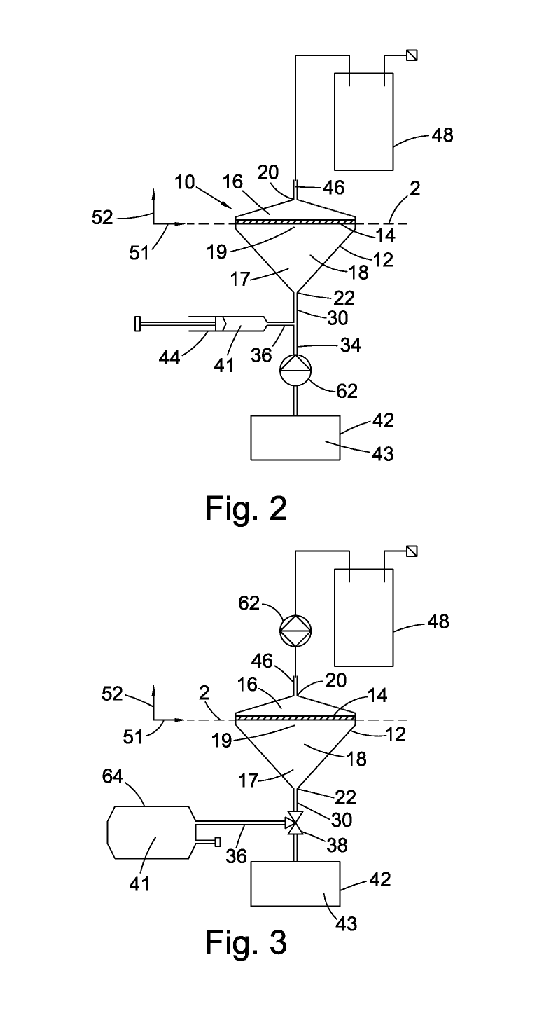

Referring to FIG. 2, an embodiment 60 of an apparatus according to the invention comprises a filter device 10 as described with reference to FIG. 1 and a fluidic system in fluid communication with the filter device, the fluidic system comprising:

(i) a collection device 44 configured to receive a liquid containing the species from the first port 22, by means of a fluidic pathway 30 leading from the port 22 and a fluidic pathway 36 from the collection device 44,
(ii) a first reservoir 42 configured to contain a second fluid 43 and a first fluidic pathway 34, 30 from the first reservoir to the first chamber,
(iii) a second reservoir 48 configured to retain liquid from the filter device 10 and a second fluidic pathway 46 from the second chamber 16 to the second reservoir 48, and
(iv) a fluidic actuation means provided in the first fluidic pathway, here comprising a pump 62.

The embodiments 60 and 70 are preferably oriented as shown in FIGS. 2 and 3, with the filter 14 wholly or mainly above the first chamber 18, such that the second region 19 proximal to the filter lies above the first region 17 comprising the port 22 as referenced by horizontal (51) and vertical (52) directions as shown in the figures.

The reservoirs may be rigid or semi-rigid vessels vented by means of breathers or may be collapsible, for example bags. Such bags may be formed from materials as known in the art and as used in cell culture, for example FEP (Fluorethylene Polymer) or polyethylene (PE), such as ultra-low density PE and may have a volume chosen according to the volume of suspension to be processed, for example a volume in the range 1 ml to 101, 10 ml to 11, 25 ml to 250 ml, or in the range 10 ml to 100 ml, 25 ml to 250 ml, 50 ml to 500 ml. Here the source of suspension 41 to be filtered is shown as the same as the collection device, namely syringe 44, connected to the inlet fluid pathway 30 by a fluid pathway 36. In use the suspension 41 is introduced into the first chamber 18 by the syringe 40. Wash liquid is flowed under positive pressure by the pump 62 from the reservoir 42 through the suspension, through the filter 14, and out through the outlet port 20 to be retained in the waste reservoir 48. When the suspension has been washed, it may be unloaded through the first port 22 back to syringe 44. Alternative fluid actuation means as described above may be used in place of the pump 62, and that a separate source of suspension from the collection device may be provided in the manner of FIG. 1.

Referring to FIG. 3, an embodiment 70 of an apparatus according to the invention comprises a filter device 10, and a fluidic system in fluid communication with the filter device comprising:

(i) a collection device 64 in fluid communication with the first port 22 by means of a fluidic pathway 30, a fluidic pathway 36 from the collection device 64, and valve means 38,
(ii) a first reservoir 42 configured to contain a second fluid 43 and a first fluidic pathway 34, 30 from the first reservoir to the first chamber,
(iii) a second reservoir 48 configured to retain liquid from the filter device 10 and a second fluidic pathway 46 from the second chamber 16 to the second reservoir 48, and
(iv) a fluidic actuation means provided in the second fluidic pathway, here comprising the pump 62.

A source of suspension 41 to be filtered, here shown as a bag 64, is connected to the inlet fluid pathway 30 by a fluid pathway 36 and a valve means 38, here shown as a 3-way valve. In use suspension is introduced into the first chamber 18 by aspiration by the pump 62. Wash liquid is flowed under negative pressure by the pump 62 from the reservoir 42 through the suspension, through the filter 14, and out through the outlet port 20 to be retained in the waste reservoir 48. When the suspension has been washed it may be unloaded, for example by means of reversing the pump 62, through the first port 22 back to bag 64 now functioning as a collection device or to a further collection device connected to the fluidic pathway 30, for example via valve means 38 which may be a 4-way valve in the manner of FIG. 1. Valve means 38 is used in this embodiment to control aspiration from one or both of the reservoir 42 and the bag 64.

In some embodiments 10, 60 and 70 the apparatus further comprises a power source to supply power to the one or more fluidic actuation means (pumps and syringe drivers) and valve actuators, for example in embodiment 60 the pump 62 and a syringe actuator to operate syringe 44, and in embodiment 70 the pump 62 and a valve actuator to operate valve 38. In some embodiments the apparatus further comprises a control means to control operation of one or more pumps and syringe and valve actuators. In some embodiments the control means comprises one or more power supply switches to control the power supply to the pumps and actuators. In some embodiments one or more such switches are manually operated. In some embodiments the control means comprises a computer means adapted to operate pumps and syringe and valve actuators in response to a program. In some embodiments the apparatus further comprises a data storage means in data communication with the control means, the control means being configured to read data from the data storage means and to operate one or more pumps and actuators in response to the data.

In this way the apparatuses shown in FIGS. 2 and 3 are configured to move the second fluid 43 from the first reservoir 42 to the first chamber 18 and configured to permit fluid to flow from the second chamber 16 to be retained in the second (waste) reservoir 48. In this way the apparatus is configured to wash the suspension within the first chamber 18, the washing being found to be particularly efficient owing to the tapering form of at least the lower region 17 of the first chamber.

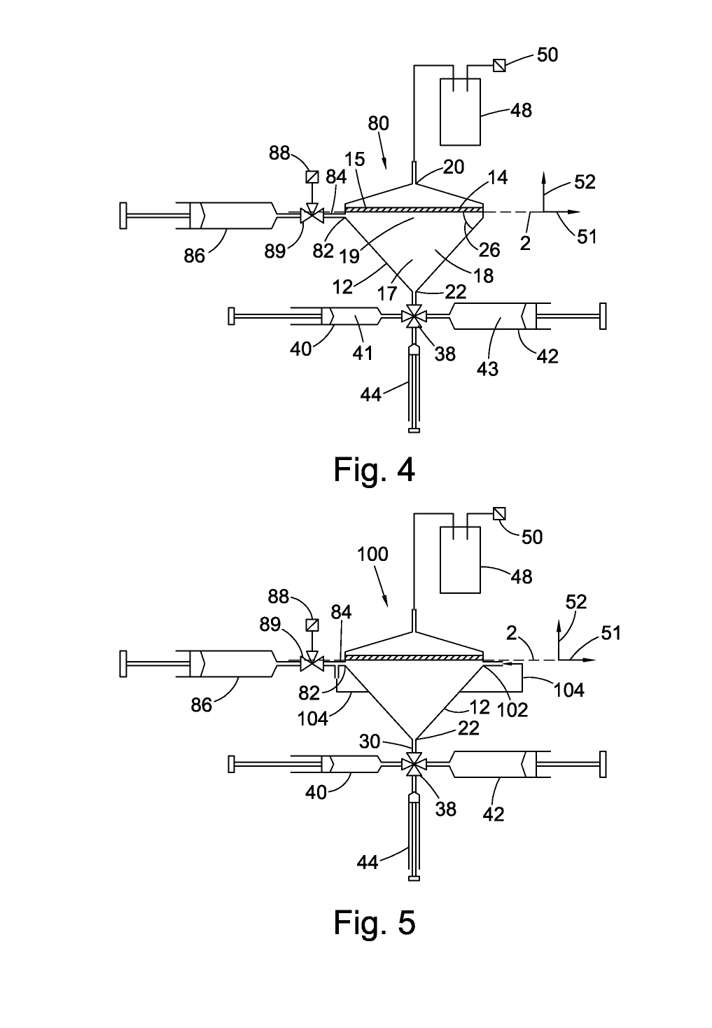

Referring to FIG. 4 an embodiment 80 of a device for filtration of a liquid containing a species in suspension comprises a vessel 12, the vessel comprising a filter 14 that divides the vessel into a first chamber 18 and a second chamber 16, the first chamber comprising a first region 17 distal to a first end 15 of the filter and a second region 19 proximal to the said first end of the filter, the vessel being provided with

(i) a first port 22 opening to the first region 17 of the first chamber, the first port being configured to permit exit of the liquid containing the species from the first chamber,
(ii) a second port 82 opening to the second region 19 of the first chamber, and
(iii) an outlet port 20 opening to the second chamber 16.

In this embodiment the filter device 80 is preferably oriented as shown in FIG. 4, with an axis 2 of the device parallel to the filter being oriented substantially horizontally such that the second region 19 proximal to the filter lies above the first region 17. In the following, the location and function of the components of the apparatus will be referred to in the orientation shown in FIG. 4, with the first region 17 of the first chamber referred to as the lower region, the second region 19 as the upper region, the first port 22 being referred to as the lower port and the port 82 as the upper port.

In some embodiments upper port 82 is connected to a hydrophobic breather adapted to allow gas to pass through it while preventing liquid from passing through it. In some embodiments the upper port 82 is connected to the breather via a valve. In this way the upper port allows gas to enter the first chamber via port 82 when the chamber is to be unloaded via port 22. In some embodiments the upper port is connected to a source of gas, for example contained within a reservoir. In some embodiments the upper port is connected to a fluidic pathway leading to the atmosphere.

In the embodiment shown in FIG. 4 the upper port 82 is configured to permit influx of a wash liquid, the upper port being provided in an upper region 19 of the first chamber, so being located closer to the filter 14 than the lower port 22. Preferably the upper port is adjacent to a first end 15 of the filter as shown in FIG. 4, and according to the embodiment may open to the wall of the first chamber either within a substantially vertical region of the wall or within a sloping region. An embodiment 90 of an apparatus comprising filter device 80 comprises a fluidic pathway 84 connected to port 82, and a source 86 of a wash liquid here shown as a syringe but which may comprise another form of reservoir together with fluid actuation means as described previously for other liquid sources. In embodiment 90 the inlet fluidic pathway 84 is shown connected to the source 86 via a valve means 89, and optionally openable to a breather 88 via the valve means 89. In some variants of this embodiment port 82 may be configured to permit influx of fluid but not passage of a species is suspension, for example, port 82 may comprise a filter in the fluidic pathway through it.

In use, a suspension may be introduced into the first chamber via port 22 as before, and in some embodiments may be introduced via port 82. The suspension may then be washed by upward flow through port 22 from wash liquid source 42. Wash liquid may simultaneously be introduced into the first chamber via port 82. The wash liquid introduced through port 82 may be the same as that introduced through port 22 or in some embodiments may be different. Sources 42 and 86 may be a common source in some embodiments with a fluidic pathway leading to each of ports 22 and 82. Species being washed are lifted within and from the first region 17 by flow of wash liquid through port 22, while wash liquid from port 82 tends to flow over the surface of filter 14 before moving through it, this flow tending to prevent the species from coming into the vicinity of the filter. In this way the tendency of the filter to capture the species and to clog is reduced. In some embodiments of the filter device 80, a recirculating flow of the species may be set up, rising from the base of the first region entrained in wash liquid from port 22, then having a horizontal component of motion through the second region and across the top of the first chamber resulting from flow from port 82. This results in efficient washing and high rates of recovery of the species.

To unload the first chamber, suspension may be unloaded to collection device 44 by opening valve 89 to the breather 88, so allowing air to enter as the first chamber is emptied.

Referring to FIG. 5 again referring to the device in the orientation as shown with reference to an axis 2 of the device parallel to the plane of the filter being oriented substantially horizontally, with the first region 17 of the first chamber being referred to as the lower region and the second region 19 as the upper region, an embodiment 100 of a filter device according to the invention comprises features as in embodiment 80 in FIG. 4, the first chamber 18 further comprising a second upper port 102 connected to a third flow pathway 104, both upper ports being configured to permit influx of a wash fluid, the first and second upper ports being provided in an upper region 19 of the first chamber closer to the filter 14 than the lower port 22. The principle of operation of the filter device 100 is as for the device 80 and apparatus 90, now with wash liquid flowing over the filter from the two upper ports, so creating a more symmetrical flow pattern in the vicinity of the filter. It will be understood that further such upper ports may be provided, and that the vessel 12 may have a range of shapes in plan view at the level of the filter, such as circular, oval, lozenge or rhomboid with the ports 82, 102 for example located at apices of the cross-sectional shape of the first chamber. In some embodiments the sideways liquid flow from ports 82, 102 may serve primarily to limit the proximity of the species to the filter, and only secondarily in washing the suspension, the majority of the flow from these ports being drawn through the filter without contacting the bulk of the suspension. In some embodiments the sideways flow may be provided by recycling liquid from the waste reservoir 48, by means of a fluidic pathway comprising a pump means, leading from the waste reservoir to fluidic pathways 84 and 104. In this way wash liquid is conserved while clogging of the filter and loss of species to the filter is reduced.

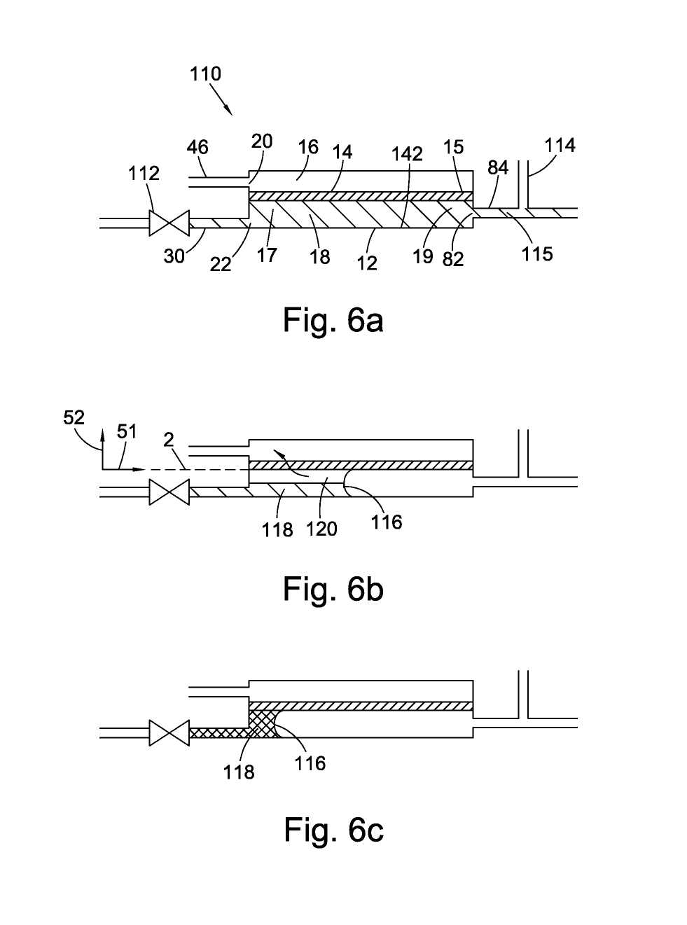

Referring to FIGS. 6a to 6c, an embodiment 110 of a filter device is shown and a method according to the invention for volume reduction of a suspension is illustrated. The filter device 110 comprises a vessel 12 comprising a filter 14 that divides the vessel into a first chamber 18 and a second chamber 18, the first chamber comprising a first region 17 distal to a first end 15 of the filter and a second region 19 proximal to the said first end, the vessel being provided with

(i) a first port 22 opening to the first region 17 of the first chamber, the first port being configured to permit exit of the liquid containing the species from the first chamber,
(ii) a second port 82 opening to the second region 19 of the first chamber, the second port being configured to receive a liquid containing the species,
(ii) an outlet port 20 opening to the second chamber 16.

In some embodiments an apparatus comprising the filter device 110 comprises a flow-cut off means connected to the outlet fluidic pathway 30. In some embodiments the flow cut-off means is a cut-off valve 112. In some embodiments the flow cut-off means comprises a fluidic actuation means configured such that fluid flow into it is prevented while the fluidic actuation means is not activated. In some embodiments the flow cut-off means comprises a collection device such as a syringe.

In some embodiments the device 110 comprises a wall 142 distal to the filter and the first port 22 to the first chamber opens to or adjacent to the wall 142. The device is preferably oriented such that the first chamber 18 lies substantially or wholly below the filter 14, and may be oriented such that axis 2 parallel to the plane of the filter 14 is substantially horizontal as shown with respect to horizontal axis 51. The device 110 may form part of an apparatus configured such that the device is oriented with the first chamber 18 substantially or wholly below the filter. In some embodiments the apparatus is configured such that first port 22 opens to a point near the lowest point of the first chamber.

The vessel 12 may have a range of shapes in plan, for example rectilinear, lozenge shaped or curved. The first and/or the second chamber may taper towards one or more of the ports 22, 82 and 20. In the embodiment shown the second port 82 is configured to introduce both of a suspension and a second immiscible fluid into the first chamber. The first port 22 is configured as an outlet port for concentrated suspension. A source of a second immiscible fluid is connected to the inlet fluidic pathway 84 by second inlet pathway 114.

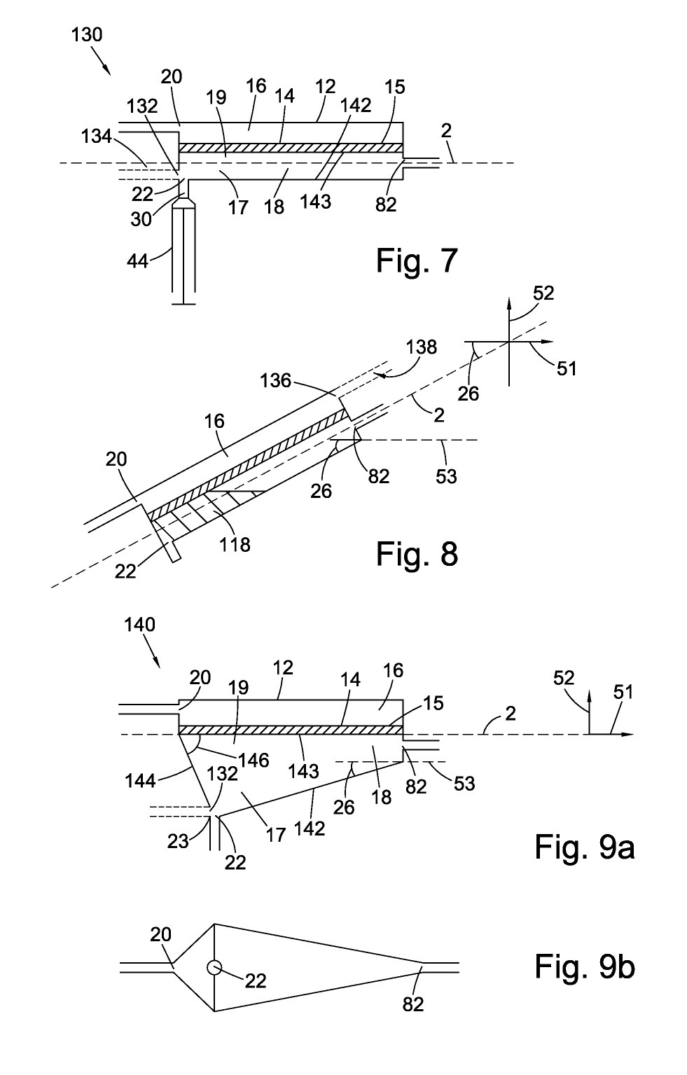

In some embodiments the second port 82 may be provided adjacent to the wall 142 of the first chamber distal to the filter. In some embodiments one or both of the first and second ports may open to the distal wall 142, for example in the manner shown for port 22 in FIG. 7.

According to the embodiment the immiscible fluid may be a gas or may be a liquid immiscible with the suspension. For example, for processing an aqueous suspension the immiscible liquid may be a non-aqueous liquid and vice-versa. The gas may be air or another gas, for example chosen to maintain conditions within the suspension, such as an inert gas, an oxygen-free gas such as pure nitrogen, a gas mixture containing oxygen, a gas mixture containing carbon dioxide such as a CO2/O2/N2 mixture to maintain the pH pf a bicarbonate-buffered liquid, as for example in the case of a cell suspension. The gas may be humidified. The filter 14 is adapted such that in use when wetted with the liquid of the suspension the immiscible fluid does not pass through it. For example, for volume reduction of an aqueous suspension a hydrophilic filter or a substantially hydrophobic filter first wetted with aqueous liquid may be used, with the immiscible fluid being a gas. If the immiscible fluid is a non-aqueous liquid then a hydrophilic filter is preferably used to avoid possible wetting of the filter with the non-aqueous liquid. The immiscible fluid will have a breakthrough or bubble pressure for passage through the filter into the second chamber. In some embodiments the apparatus and method are adapted such that the maximum applied pressure across the filter is less than the breakthrough or bubble pressure. In the following description the immiscible fluid will be referred to as a gas, but it will be understood that similar principles will apply in the case of an immiscible liquid with modifications to the configuration of the apparatus as will be apparent to a skilled person.

An embodiment of a method of operation of the filter device 110 comprises some or all of the following steps. Referring to FIG. 6a, a suspension 115 is introduced into the first chamber 18 and the cut-off valve 112 is shut; alternatively in some methods cut-off valve 112 may first be shut and the suspension may then be introduced, liquid passing through the filter from the first chamber if the volume introduced is greater than the volume of the first chamber. Referring to FIG. 6b, gas is then introduced into the first chamber via port 82 from fluidic pathway 114 to produce a meniscus 116. As gas is introduced, liquid from the suspension is displaced through the filter as shown by the arrow resulting in concentration of the suspension. In the case where the device is oriented as in FIGS. 6a to 6c, with the filter 14 substantially above the first chamber 18, and in which the suspension is such that sedimentation of species occurs, a concentrated layer 118 may form preferentially towards the base of the first chamber (the distal wall 142) ahead of the meniscus, with a less concentrated layer 120 above it, from which liquid passes through the filter. As the meniscus moves further through the first chamber the suspension is concentrated still more as shown in FIG. 6c. At a chosen degree of volume reduction, for example controlled by introduction of a known amount of gas, the process may be stopped. As the meniscus nears or reaches the end of the first chamber and the liquid flow pathway from the concentrated suspension to the filter becomes more limited, the pressure drop across the filter will tend to increase and, in the case where a constant pressure is applied to the gas, movement of the meniscus tends to slow and may stop. If gas is driven by a constant displacement fluidic actuation means such as a syringe or a constant displacement pump pressure will begin to build up in the fluidic pathway to port 82. In some embodiments where introduction of gas is driven by a constant driving pressure the volume reduction process may self-terminate at the point that the pressure drop across the filter reaches the driving pressure. Following volume reduction the cut-off valve 112 is opened and the concentrated suspension is unloaded from the first chamber through port 22 and fluidic pathway 30.

In some embodiments, a source of wash liquid may be connected to one or both of inlet flow pathway 84 or outlet flow pathway 30 and a method of operation may comprise the further step of introducing a wash liquid into the first chamber.

In some embodiments, the wash process may include the step of volume reduction before wash liquid is introduced, and may include repeated alternating steps of introducing a wash liquid and of volume reduction. Washing in this way may require less wash liquid to achieve a given degree of washing as compared with simply flowing the wash liquid through the suspension, as the initial liquid in the suspension is partially removed to waste rather than being diluted. A step of mixing of the concentrated suspension with the wash liquid, for example by moving adjoining slugs of liquid to and fro in the first chamber, may be included.

Referring to FIG. 7 a further embodiment 130 of a filter device according to the invention is described in the orientation shown in the figure, with axis 2 of the device parallel to the plane of the filter being substantially horizontal. The embodiment 130 comprises a vessel 12, the vessel comprising a filter 14 that divides the vessel into a first chamber 18 and a second chamber 16, the first chamber comprising a first region 17 distal to a first end 15 of the filter and a second region 19 proximal to said first end of the filter, wherein the second region is above the first region, the vessel being provided with

(i) a lower port 22 opening to the first region 17 of the first chamber, the lower port being configured to permit exit of the liquid containing the species from the first chamber,
(ii) an upper port 82 opening to the second region 19 of the first chamber, and
(iii) an outlet port 20 opening to the second chamber 16.
and wherein the first chamber comprises a lower wall 142 and a planar upper wall 143 spaced apart from the lower wall, wherein the filter is a planar filter 14 forming part of the upper wall 143.

As shown in FIG. 7 an embodiment of the device 130 comprises a collection device 44 directly connected to the lower port and configured to receive a liquid containing the species from the lower port 22, by means of fluidic pathway 30. In this embodiment the collection device 44 provides a flow cut-off means connected to the outlet fluidic pathway 30. In this way the outlet fluidic pathway 30 is short and does not include a cut-off valve 112 or other valves or junctions, so reducing risk that concentrated suspension may be held up in such components or lost by adsorption to surfaces within the fluidic pathways connecting them. The device is oriented such that the lower port 22 opens to the lower wall of the first chamber and is configured to allow unloading of the suspension by means of a downward component of flow direction, allowing gravity-assisted flow towards the lower port. This feature is especially useful for concentrated suspensions.

In some embodiments the first chamber is provided with a further port 132 opening to the first region 17 of the first chamber, the further port being configured to receive one or both of a liquid containing the species and a wash liquid.

In some embodiments the further port 132 is provided at or adjacent to the base of the first chamber. The device may be configured to introduce a wash liquid to the first chamber through the further port 132 and a fluidic pathway 134 separate from the fluidic pathway 30. This allows a direct and short fluidic pathway 30 from the first lower port to be directly connected to the collection device without a fluidic junction or valve, so assisting complete unloading of the filter device and reducing the possibility of loss of species to the surfaces of the fluidic pathway or to hold-up at junctions or discontinuities in the surfaces of the pathway 30.

Referring to FIG. 8, in some embodiments the filter device 130 of FIGS. 6a-c and FIG. 7 is oriented at an angle to horizontal in use, as shown in FIG. 8 by reference to an axis 2 of the device being at an angle 26 to horizontal axis 51. This offers advantages including (i) sedimentation of species from the suspension may concentrate species adjacent to the first port 22 and (ii) unloading through the port 22 can be more efficient than with the device oriented with the base of the first chamber being horizontal. Optionally for a device adapted to operate at an angle as shown in FIG. 8 a second chamber outlet port 136 is provided at the upper end of the second chamber 16, to allow priming of the second chamber with liquid and for air to escape through the fluidic pathway 138. Optionally both a first and a second filtrate outlet ports 20 and 136 may be provided to assist in priming the second chamber. A device may be adapted to present the filter chamber at a chosen angle, for example the device may comprise a sloping chamber or vessel within a body component having support means defining a plane on which the device may be mounted substantially horizontally.

An apparatus comprising a filter device as described herein may be configured to present the device at an angle to the horizontal, for example comprising a surface on which that device is mounted, the surface being at an angle to the horizontal when the apparatus is positioned ready for use.

Optionally the filter device 130 may be tilted in use from a first value of angle 26, for example horizontal, to a second angle, for example an angle in the range 0 to 60 degrees, and in some embodiments an angle in the range 25 to 45 degrees. In some embodiments an apparatus comprises a tilting means adapted to change the orientation of the filter device. For example, the filter device may be mounted on an actuator adapted to tilt the device to achieve one or more of a slope with port 22 lowermost as shown in FIG. 8, a slope in the opposite sense such that port 82 is lowermost, or either one following the other. In one method of use a suspension is introduced into the first chamber via the second port 82 and is washed by wash liquid also introduced via port 82 while the device is horizontal, or in some embodiments while the device is tilted such that port 82 is at a lower position than port 22. The device may then be tilted such that port 22 is lower than port 82 to allow sedimentation of species towards port 22. Optionally volume reduction may be achieved by introducing a gas via port 82 as described for FIGS. 6a-c. The suspension may then be unloaded through port 22, for example to a collection device (not shown).

FIG. 9a shows a cross section along the long axis of a further embodiment 140 of a device according to the invention, and FIG. 9b shows a plan view cross section of the top of the first chamber parallel to the filter.

Referring to FIGS. 9a and 9b, a further embodiment 140 of a filter device according to the invention comprises a vessel 12, the vessel comprising a filter 14 that divides the vessel into a first chamber 18 and a second chamber 16, the first chamber comprising a first region 17 distal to a first end 15 of the filter and a second region 19 proximal to the said first end of the filter, the vessel being provided with

(i) a first port 22 opening to the first region 17 of the first chamber, the first port being configured to permit exit of the liquid containing the species from the first chamber,
(ii) a second port 82 opening to the second region 19 of the first chamber, and
(iii) an outlet port 20 opening to the second chamber 16.
and wherein the first chamber comprises a distal wall 142 and a proximal wall 143 spaced apart from the distal wall, wherein the filter forms part of the proximal wall 143, and at least a portion of distal wall 142 of the first chamber 18 forms a first angle 26 to the plane of the filter.

In some embodiments the first angle is between approximately 3 and approximately 30 degrees, and in some embodiments the first angle is between approximately 30 and approximately 85 degrees.

In some embodiments the filter 14 is substantially planar. The plane of the filter is a plane characterising a planar filter when in situ in the device, for example the plane encompassing at least the majority of the perimeter of the filter or a plane forming the mean position of a corrugated, rippled, dimpled or bowed filter.

In some embodiments at least a portion of the distal wall 142 forms a first angle 26 to an axis 2 parallel to the filter.

In this embodiment the first chamber tapers towards the first port 22, the term ‘tapers towards’ having a meaning as defined herein. The first chamber also tapers towards the second port 82. In use, such a taper reduces or avoids trapping zones for air in priming and dead zones for introduction of suspension, and allows the meniscus to expand gradually on introduction of gas as described below. In variants of this embodiment the taper may take a range of forms as defined herein.

In variants of this embodiment the first chamber has a rectilinear, triangular, semicircular, or semi-oval shape in the third cross-section perpendicular to the long axis 2 of the device. In variants of this embodiment the vessel 12 has a rectilinear, triangular, semicircular, or semi-oval shape.

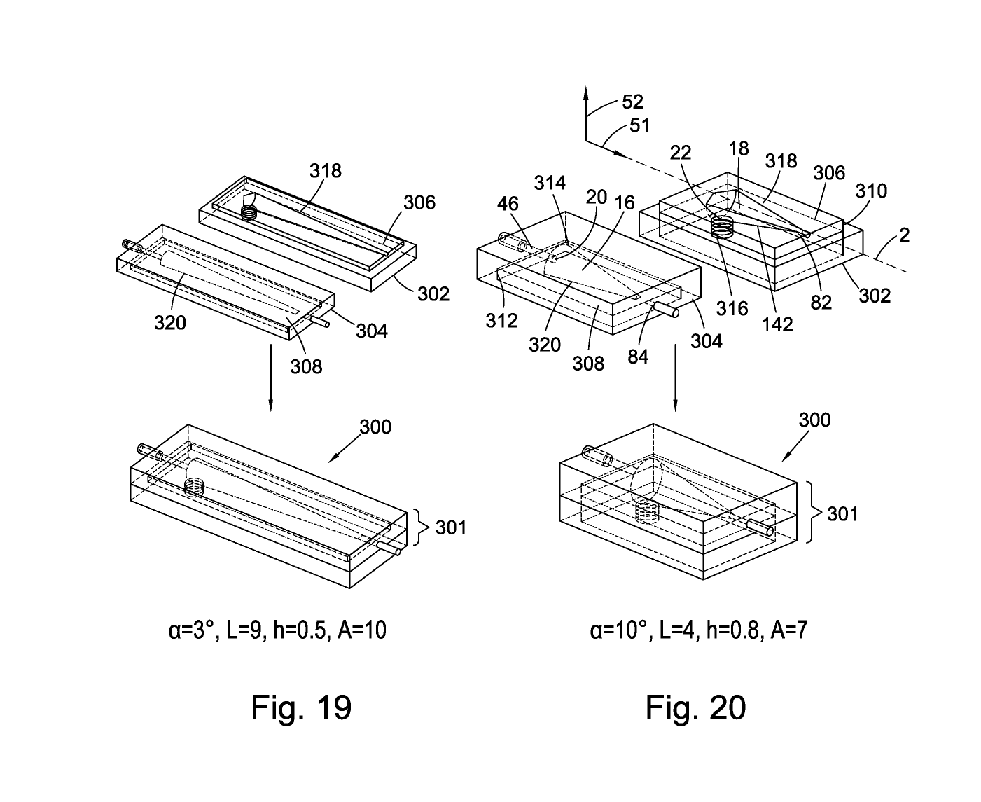

An embodiment 400 according to FIGS. 9a and 9b is shown in FIGS. 19 and 20 wherein the first chamber has a shape formed from two truncated half-cones having a join line at their bases, the lower port 22 opening to the lowest point of that join line. In embodiment 400 the third cross-sectional shape perpendicular to the axis 2 is a semicircle, whose radius decreases as the first chamber tapers towards port 82.

In FIG. 9a the filter is shown as a planar filter forming substantially the whole of the proximal wall 143, though it will be recognised that the filter may occupy only a portion of the proximal wall and the filter may be only approximately planar, for example having regions of curvature parallel to or perpendicular to the axis 2. The first chamber has an end wall 144 inclined at an angle 146 to the plane of the filter. In this way the first chamber presents a substantially triangular vertical cross-section as shown.

The device is preferably oriented as shown in FIG. 9a, with the filter 14 substantially or wholly above the first chamber 18. In some embodiments the axis 2 of the device, parallel to the filter, may be substantially horizontal. In this way the distal wall 142 is the lower wall of the vessel and slopes downwards towards the lower port at an angle 26 to the horizontal as indicated by horizontal axis 51 and axis 53 parallel to axis 2. The first port 22 is thereby located substantially at the lowest point of the first chamber. The first region 17 is referred to as the lower region and the first port 22 as a lower port, the second region 19 is referred to the upper region and the second port 82 as an upper port.

In this way the first chamber provides a slope within at least the lower region 17 of the first chamber 18 towards the lower port 22 to permit efficient unloading of suspension. The first chamber may comprise a region of convex curvature where the port 22 opens to the wall 142 of the first chamber in order to provide a continuous surface from the wall into the fluidic pathway 30. The fluidic pathway 30 may comprise a tapering section leading from the port 22, one or more cross-sectional dimensions of the pathway decreasing with distance from the port.

Optionally a further lower port 132 may be provided as described for the embodiment 130 in FIG. 7.

In some variants of this embodiment one or both of ports 132 and 82 may be configured to permit influx of fluid but not passage of a species is suspension, for example, a port may comprise a filter in the fluidic pathway through it.

The shape of the second chamber 16 is shown as a simple rectilinear cross section in FIG. 9a, but may be other shapes, for example it may be substantially a mirror image of the first chamber as reflected in the plane of the filter as shown in FIGS. 19 and 20.

In this way the device provides a vessel 12 comprising a filter 14 dividing the vessel into a first chamber 18 and a second chamber 16, the first chamber comprising an upper filtration region 19 having the filter forming at least part of a wall 143 of region 19, and a lower sedimentation region 17, comprising impermeable walls 142, 144 and having a first port 22 provided opening to the sedimentation region. In some embodiments the first port 22 is provided opening at or adjacent to the lowest point 23 of the sedimentation region.

In a method of use with the device oriented as in FIG. 9a with the filter substantially above the first chamber 18, a suspension is introduced into the first chamber, either through the lower port 22 or the upper port 82. The suspension is then washed by introducing a wash liquid through lower port 22 (or a second lower port 132, where present), through port 82, or through both ports 82 and port 22 (or port 132) simultaneously. Gas may then be introduced through port 82 to achieve volume reduction in the manner described with reference to FIGS. 6a-c. On introducing gas, a meniscus is formed that may contact the filter and the lower wall 142 and has a profile determined in part by the gas/liquid/solid contact angles on the filter and lower wall. The meniscus advances from port 82 through the first chamber, while liquid is displaced through the filter into the second chamber and through port 20. The suspension is concentrated on the liquid side of the meniscus, that is the side distal from port 82, i.e. proximal to port 22. The concentrate may then be unloaded through the port 22, assisted by gravity. In some embodiments the gas may be introduced as a series of separate bubbles, each bubble being retained in the first chamber and effecting an increment in the volume reduction process. In some cases the bubbles may coalesce. This may leave one bubble that is larger than the rest and plays the primary part in the volume reduction process.

In this way the device 140 is adapted for efficient washing, volume reduction and unloading of the suspension. According to the type of filter, the device 140 is adapted to separate a first species from components in solution, or a first species in suspension from a second species in suspension that is able to pass through the filter.

The angle 26 may be chosen to achieve some or all of (i) stability of the meniscus; (ii) efficient washing of the suspension: as will be shown by the examples, washing in this embodiment is more efficient when wash liquid is introduced from the port 22 at the base of the first chamber upwards through the suspension than when introduced through port 82; (iii) efficient volume reduction and draining when the concentrate is unloaded: a steeper slope of the wall 142 near the port 22 is expected to assist flow of concentrate into the port, and (iv) a convenient overall size and shape for the filter device: a larger angle will result in a shorter, deeper, lower aspect ratio device that is easier to make and to include in surrounding apparatus. The angle 146 will in general be greater than the angle 26. In some embodiments the angle 26 is in the range 3 to 30 degrees. In some embodiments the angle 26 is in the range 3 to 15 degrees. One preferred value of the angle 26 is 10 degrees, as shown in the examples, but other values may be chosen depending on the importance of each of the factors (i) to (iv) above in a particular embodiment or application.

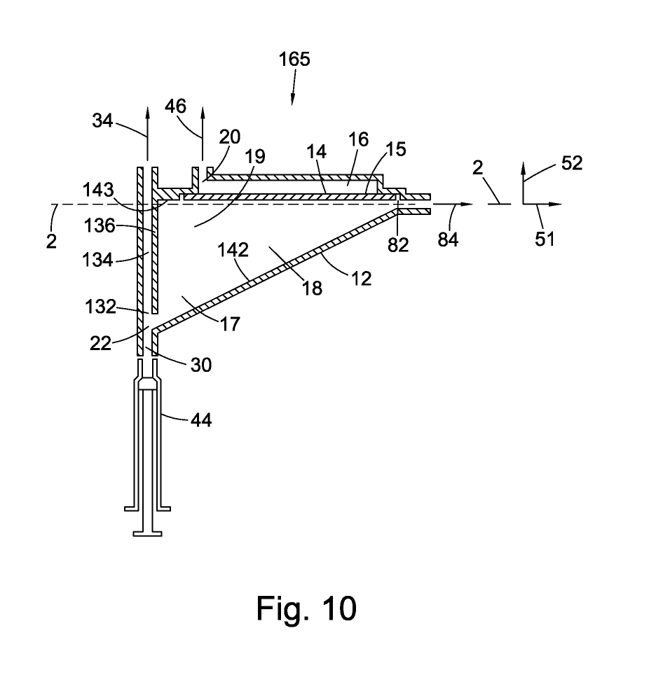

Referring to FIG. 10, a further embodiment 165 of a filter device according to the invention has features as shown in FIG. 9a, and in which a further port 132 is provided opening to the first region 17, and a fluidic pathway 134 provided in the form of a conduit formed within the device, connected to port 132. The collection device, here shown as a syringe 44, is connected directly to the first port 22 via fluidic pathway 30. This device has the advantage that port 132 may function as an inlet for wash liquid and in some embodiments for the suspension, while port 22 is used only for unloading the suspension to the collection device.

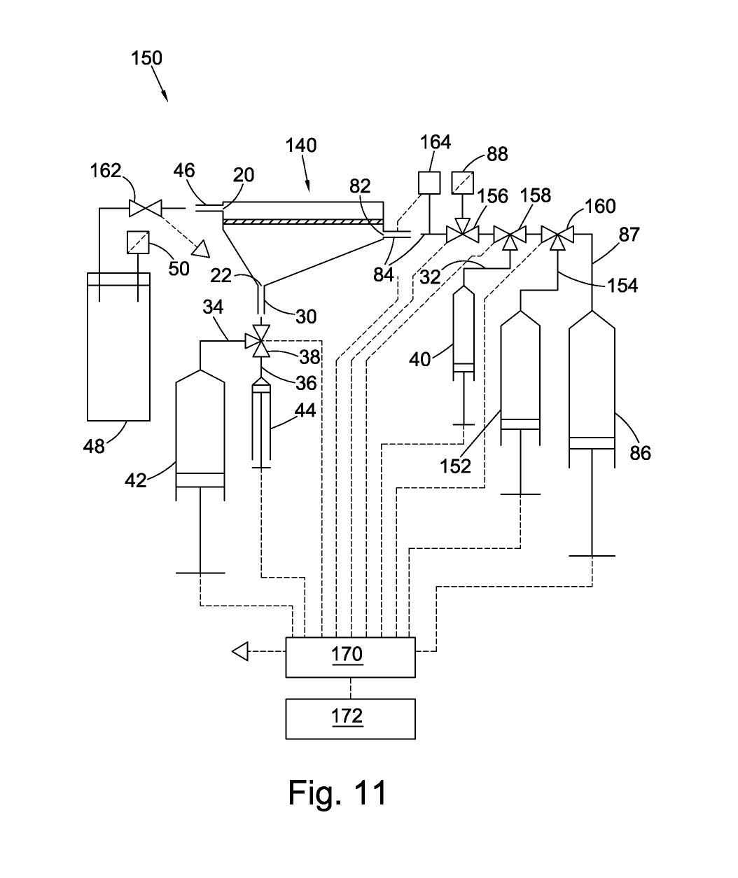

The embodiment 165 may be used for example with a fluidic system as shown in FIG. 11, with ports 132, 82 and 20 connected to fluidic pathways 34, 84 and 46 respectively. The operation of the device 165 is as described for the device in FIG. 11, with the difference that the source of wash liquid 42 is now connected to the further port 132, and valve 38 and its operating step in the method are omitted. It will be understood that the filter device 165 may be provided with a cut-off valve connected to fluidic pathway 30 instead of the direct connection to syringe 44. In some embodiments the filter device and apparatus comprising it are configured to allow the suspension to be introduced into the first chamber via port 132 and conduit 134.

Referring to FIG. 11, an embodiment 150 of an apparatus comprising the filter device 140 is shown with common parts with previous embodiments having the same numerals. The apparatus comprises an inlet fluid pathway 84 leading to upper port 82, connected to a source of cells 40 via valve means 158, a source of gas 152 connected to port 82 via gas fluidic pathway 154 and valve means 160, a source of wash liquid 86 connected to port 82 via wash liquid fluidic pathway 87 and valve means 160, and optionally a breather 88 connected to port 82 via valve means 156. Optionally a pressure gauge 164 is provided to measure pressure in the inlet fluidic pathway 84. An outlet fluidic pathway 46 is connected to the filtrate port 20 and to a waste container 48 via valve means 162. A lower port fluidic pathway 30 is connected to the lower port 22 and via valve means 38 to a collection device 44 and to a source of wash liquid 42 via a wash liquid fluidic pathway 34. In some embodiments device 140 comprises a second lower port 132 as shown in FIG. 9a and the wash liquid fluidic pathway 34 is connected to port 132. It will be understood that while the sources of fluids 40, 42, 86 and 152 are shown as syringes, the apparatus of the invention is not limited to specific types of source, reservoir or fluid actuation means and other reservoirs such as rigid or flexible containers, and fluid actuation means such as pumps, gas pressure or gravity may be used in some embodiments.

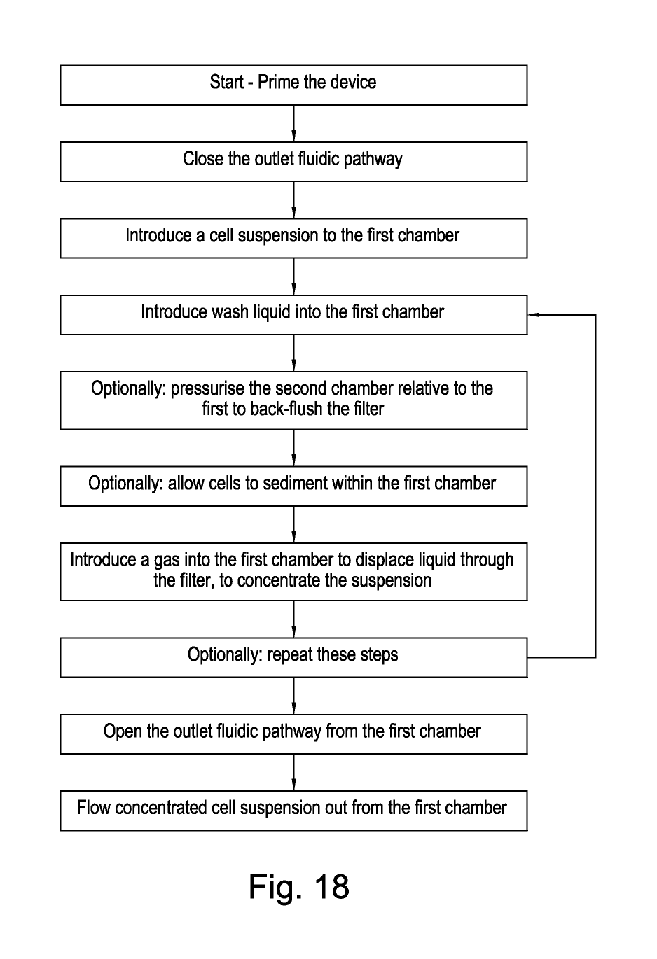

A method of use may follow the steps as shown in FIG. 18, for the example case where the species is cells.

1. Prime the filter device: flow wash liquid from source 42 through port 22 to fill the first chamber, optionally venting air via valve 156 and breather 88, and through the filter to vent air into the waste container 48.
2. Close the outlet fluidic pathway 30 from the first chamber by means of valve 38.
3. Flow cell suspension into the first chamber from source 40 and port 82.
4. Flow wash liquid into the first chamber from source 42 via port 22 and via filter 14 to the waste container 48. Optionally flow wash liquid from source 86 via port 82 simultaneously.
5. Optionally: pressurise the second chamber 16 relative to the first chamber 18 to back flush the filter.
6. Optionally: allow cells to sediment within the first chamber. In some embodiments of the method this step may be done before step 5 (where present).
7. Introduce gas from source 152 into the first chamber via gas fluidic pathway 154 and valve 160 to displace liquid through the filter and effect volume reduction in the first chamber.
8. Optionally: steps 4 to 7 may be repeated in order to achieve diafiltration by means of sequential volume reduction and dilution. In some methods step 7 is done first and followed by steps 4 to 6.
9. Open the outlet flow pathway 30, 36 from port 22 to the collection device—namely syringe 44—and flow concentrated cell suspension out from the first chamber by withdrawing the syringe plunger. Pressure equilibration in the first chamber is optionally done by opening valve 156 to breather 88. In some embodiments pressure equilibration may be achieved by introducing gas from source 152 via fluidic pathway 84 and valve 160.

In some embodiments of the apparatus valve 38 might be replaced by a T-junction and fluid flow in pathways 34 and 36 is controlled by controlling actuation of syringes 42 and 44.

In some embodiments pressure gauge 164 is provided to monitor pressure in the inlet fluidic pathway 84, for example to monitor pressure as gas is introduced into the first chamber for volume reduction.

Some embodiments comprise a control means 170 adapted to control the fluid actuation means and the valves, and optionally to read information from sensors such a pressure sensor 164, as illustrated by dotted lines in FIG. 11. The control means 170 comprises computer control means as known in the art, and preferably reads data from a data storage means 172 in order to control operation of the apparatus. Such data may comprise data about the suspension to be processed and may be specific to an individual batch of suspension within the source container 40. For example, in the case that the suspension is a cell suspension for administration to a patient, the data may comprise data related to the cell suspension, the patient, or both. The data storage means 172 may be a local data store or a remote data store accessed via a data network. The control means may exchange data with one or more remote computer systems. In some embodiments the control means comprises means to monitor one or more parameters associated with the process, such as temperature, and to record the parameters in a data storage means. In some embodiments the control means is configured to provide data resulting from the process in a report or to communicate such data to a remote computer system.

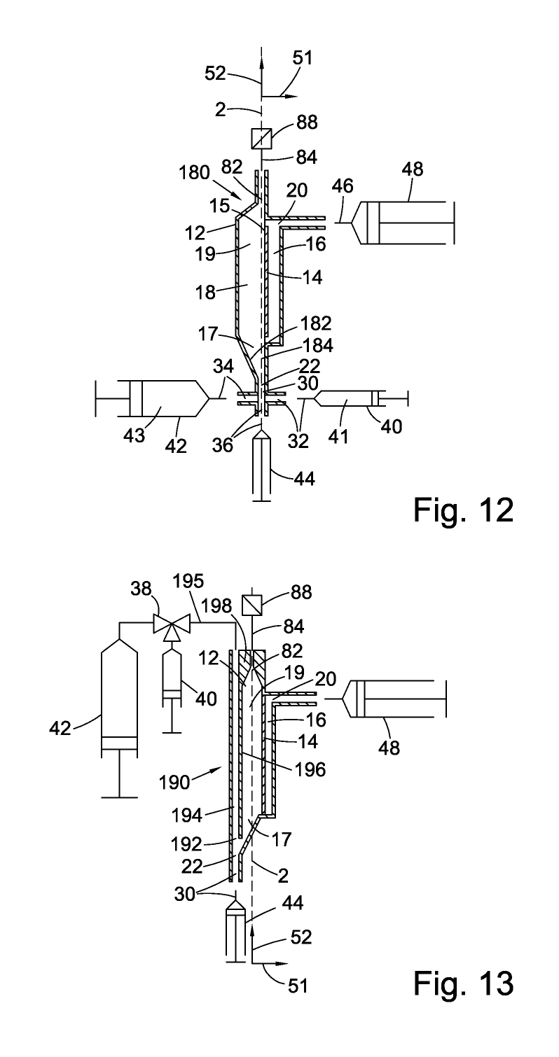

Referring to FIG. 12 a further embodiment 180 of a filter device according to the invention comprises a vessel 12, the vessel comprising a filter 14 that divides the vessel into a first chamber 18 and a second chamber 16, the first chamber comprising a first region 17 distal to a first end 15 of the filter and a second region 19 proximal to the first end 15, the vessel being provided with:

(i) a first port 22 opening to the first region 17 of the first chamber, the first port being configured to receive a liquid 41 containing the species and further configured to permit exit of the liquid containing the species from the first chamber,
(ii) a second port 82 opening to the second region 19 of the first chamber, and
(iii) an outlet port 20 opening to the second chamber 16.

In some embodiments the device 180 is oriented as shown in FIG. 12, with the filter 14 oriented away from horizontal, the axis 2 of the device parallel to the filter being at an angle to vertical. In some embodiments of a method of use the axis 2 is substantially vertical.

In this way the second region 19 lies above the first. The first region 17 may be referred to as the lower region and the first port 22 as a lower port, the second region 19 may be referred to the upper region and the second port 82 as an upper port. The first port 22 is thereby located substantially at the lowest point of the first chamber.

In an embodiment the device 180 forms part of an apparatus configured such that the axis 2 and the filter 14 are oriented at and angle from horizontal, and in some embodiments are substantially vertical.

In an embodiment as shown in FIG. 12 an apparatus comprises the filter device and a source 40 of suspension 41, a source 42 of a second liquid or wash liquid 43 and collection device 44, here shown in the form of syringes, in fluid communication with the lower port 22. In some embodiments the apparatus comprises a network of two or more fluidic pathways selected from 32, 34 and 36 as shown diagrammatically in FIG. 12 and a junction between them, each fluidic pathway being connected to the first port fluidic pathway 30. The fluidic pathways may be straight or curved and may have lengths chosen to be suitable for the application, for example fluidic pathway 36 for unloading suspension from port 22 is preferably short as the suspension to be unloaded may be concentrated; fluidic pathway 32 for introducing a suspension into port 22 is preferably short but may be longer than pathway 36, as the suspension to be introduced comprises the species but may in some instances be dilute, and fluidic pathway 34 for introducing a wash liquid into port 22 may be longer as it does not contain the species. In this embodiment a flow actuation means and a waste reservoir are combined in a syringe 48 in fluid communication with the outlet port 20, allowing the device 180 to be operated by aspiration by syringe 48 in combination with operations of syringe 40 or syringe 42. It will be understood that the syringes may be replaced in some embodiments by reservoirs and fluidic actuators as described previously.

In some embodiments the device 180 comprises a vessel 12 comprising a filter 14 dividing the vessel into a first chamber 18 and a second chamber 16, the first chamber comprising an upper filtration region 19 having the filter forming at least part of a wall 143, and a lower sedimentation region 17 extending from below the lower extremity of the filter and having impermeable walls 182, 184 wherein a first port 22 is provided opening to the sedimentation region; The first port 22 is provided opening at or adjacent to the lowest point 23 of the sedimentation region.

The sedimentation region tapers towards the first port 22, having at least one of walls 182, 184 sloping towards the first port.

The sedimentation region preferably tapers towards the first port in two dimensions, and may comprise a square or rectangular pyramidal or a frustoconical shape.

In use, gas is introduced into the first chamber via second port 82 to effect volume reduction, and the meniscus of the cell suspension moves downwards within the upper region 19 of the first chamber as liquid passes through the filter. When the meniscus reaches the bottom of the filter, no more liquid can pass through it, and the volume reduction process stops. Concentrated cell suspension then remains in the sedimentation region 17. In this way the device achieves a known maximum degree of volume reduction and the risk of concentration to dryness is avoided. In embodiments with fluid actuation means that apply a chosen pressure to the gas, for example a fixed gas pressure or pressure due to gravity, the volume reduction process may be self-terminating when the pressure drop across the filter reaches the pressure of the gas.

A method of operation will be described for embodiment 190 in FIG. 13, and a method with similar steps is usable with embodiment 180. Minor variations in the method arising from the specific configuration of the fluidic system will be apparent to the skilled reader.

Referring to FIG. 13 a further embodiment 190 of a device according to the invention comprises a vessel 12, the vessel comprising a filter 14 that divides the vessel into a first chamber 18 and a second chamber 16, the first chamber comprising a first region 17 and a second region 19 wherein the second region is above the first, the vessel being provided with

(i) a first lower port 22 opening to the first region 17 of the first chamber, the first port 22 being configured to permit exit of the liquid containing the species from the first chamber,
(ii) an upper port 82 opening to the second region 19 of the first chamber, and
(iii) an outlet port 20 opening to the second chamber 16.
the filter 14 being oriented at an angle to horizontal as shown relative to the horizontal axis 51, and wherein the first chamber 18 is provided with a second lower port 192 opening to the first region 17 of the first chamber and configured to receive a liquid containing the species.

In some embodiments as shown in FIG. 13 the filter is substantially vertical.

In this embodiment the port 192 comprises an opening to a conduit 194 within the device 190. In some embodiments the device 190 comprises a body component within which vessel 12 and conduit 194 are formed. In some embodiments the conduit 194 comprises a tubular component extending through a surface of the first chamber. It is an advantage of the embodiment 190 that a separate fluidic pathway 30 is configured for unloading concentrated suspension from a fluidic pathway 195 configured for loading the suspension initially and for providing the wash liquid. In this way the pathway 30 may be short and may avoid junctions or valve means and so may reduce the risk of loss of species to the surfaces of the fluidic pathway or to hold-up at junctions or discontinuities in the surfaces of the pathway 30.

A method of use of the device 190 comprises the following steps:

1. Fill the filter device with buffer: flow wash liquid from source 42 via valve 38 through conduit 194 and port 192 to fill the first chamber, venting air via breather 88, and through the filter 14, via the second chamber 16 to the waste syringe 48.
2. Flow suspension into the first chamber from source 40 via valve 38, conduit 194 and port 192.
3. Flow wash liquid into the first chamber from source 42 via valve 38, conduit 194 and port 192 to wash the suspension.
4. Optionally: pressurise the second chamber 16 relative to the first chamber 18 to back flush the filter. Flow liquid from the second chamber through the filter into the first chamber and into conduit 194.
5. Optionally: allow cells to sediment within the first, lower region 17 of the first chamber. In some embodiments of the method this step may be done before step 4 (where present).
6. Aspirate using syringe 48 with the flow pathway 195 into port 192 closed, for example by closing valve 38 or holding the plungers of syringes 40, 42 stationary. This will introduce air through hydrophobic breather 88 into the upper, filtration region 19 of the first chamber via second port 82 while liquid is aspirated through the filter to effect volume reduction in the first chamber.
7. Optionally: steps 3 to 6 may be repeated in order to achieve diafiltration by means of sequential volume reduction and dilution. In some embodiments of a method step 6 is done first and followed by steps 3 to 5.
8. Open the outlet flow pathway 30 from port 22 to the collection device 44—and flow concentrated cell suspension out from the first chamber by withdrawing the syringe plunger. Pressure equilibration in the first chamber is done by venting through breather 88.

It will be understood that embodiments 180 and 190 may be operated by positive pressure at the inlet ports rather than aspiration at port 20, in the manner of embodiment 140 in FIG. 11, and in some embodiments the breather 84 is omitted and second port 82 is connected to a reservoir of gas.

In further embodiments a device as shown in FIG. 12 or 13 have the same features but port 82 is omitted. In such embodiments the first chamber may be filled, and the second fluid may be introduced, through one or more lower ports 22, 192. For example in embodiment 190 in FIG. 13, a source of gas may be connected to port 192 via fluidic pathway 195 and gas introduced into the first chamber from below. The gas will accumulate in the upper region 19 of the first chamber while liquid is displaced through the filter as before.

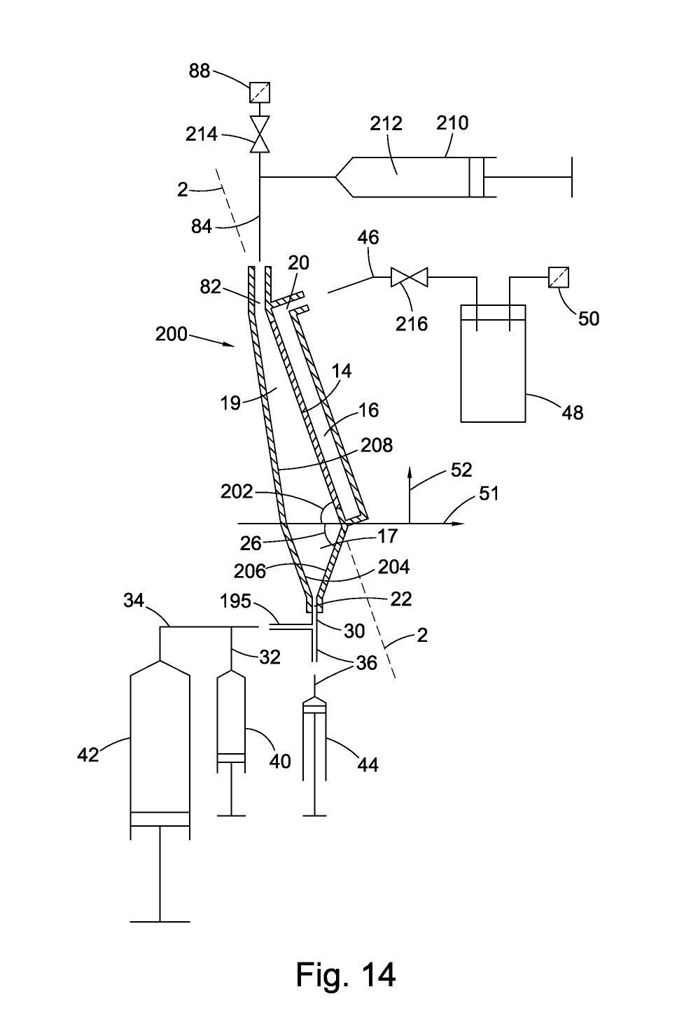

Referring to FIG. 14, an embodiment 200 of a device according to the invention comprises features substantially as for embodiment 180 wherein the filter 14 is disposed at a second angle 202 to the horizontal, the second angle being defined by the angle 202 between an axis 2 of the device parallel to the plane of the filter and horizontal axis 51 as shown in FIG. 14. According to the embodiment the angle 202 is in the range approximately 10 to approximately 90 degrees, the value shown in FIG. 14 for angle 202 being approximately 80 degrees.

Such a sloping filter gives the advantage that species that tend to sediment either on initial introduction to the first chamber, on washing or during volume reduction tend to fall away from the filter, so reducing the tendency to adsorb onto or to clog the filter.

The first chamber 18 comprises a first, lower region 17 and a second, upper region 19, the lower region 17 having one or more walls sloping at a first angle 26 to the horizontal, angle 26 having a range of values according to the embodiment as described before. First region 17 tapers towards first port 22, preferably tapering in two dimensions. Lower region 17 in some embodiments forms a sedimentation region and is adapted to permit unloading of concentrated suspension through port 22. The walls 204, 206 shown in FIG. 14 may be substantially planar walls and the sedimentation region 17 may be in the form of a rectilinear-based pyramid, or they may be portions of a substantially frustoconical wall. In embodiments where the filter 14 is a substantially planar filter, the horizontal cross-sectional shape of the lower region 17 may change from rectilinear at the level of the lower edge of the filter to circular closer to port 22. According to the embodiment the wall 204, 208 of the first chamber 18 may have a discontinuity as shown, may be smooth, or may be substantially planar in the upper region 19.

The fluidic system in fluid communication with the device 200 in FIG. 14 comprises features as described before, having common numerals and functions. The fluidic pathways 30, 36, 32, 34, and combined pathway 195 may be provided as part of the filter device 200 with appropriate connections to allow syringes 40, 42, 44 to be connected to them, or may be provided as part of a fluidic system external to the device, connection being made to fluidic pathway 30 within the device by a suitable connector means. In the fluidic system in FIG. 14 filtration and volume reduction are driven by positive gas pressure from syringe 210.

A method of use of device 200 together with the fluidic system as set out in FIG. 14 comprises the following steps:

1. Prime filter device 200: flow wash liquid from source 42 via port 22 to fill the first chamber, venting air via breather 88, and through the filter 14, via the second chamber 16 to the waste reservoir 48.
2. Flow suspension into the first chamber from source 40 via port 22.
3. Flow wash liquid into the first chamber from source 42 via port 22.
4. Optionally: pressurise the second chamber 16 relative to the first chamber 18 to backflush the filter.
5. Optionally: allow cells to sediment within the lower region 17 of the first chamber. In some embodiments of the method this step may be done before step 4 (where present).
6. Introduce gas 212 under pressure using syringe 210 through fluidic pathway 84 and port 82 into the upper region 19 of the first chamber, closing valve 214 to prevent venting through breather 88, to effect volume reduction in the first chamber.
7. Optionally: steps 3 to 6 may be repeated in order to achieve diafiltration by means of sequential volume reduction and dilution. In some embodiments of the method step 6 is done first and followed by steps 3 to 5.
8. Flow concentrated cell suspension out from the first chamber via port 22 by withdrawing the plunger of syringe 44. Pressure equilibration in the first chamber is done by venting through breather 88.

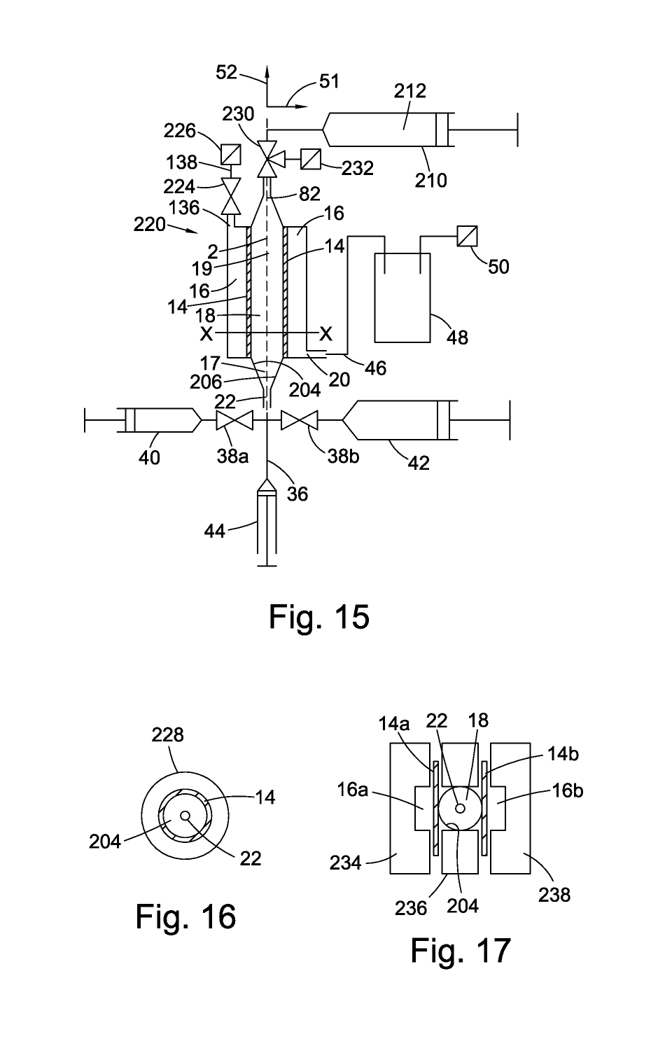

Referring to FIGS. 15 and 16, a further embodiment 220 of a filter device according to the invention comprises features as described previously with common numerals, wherein the vessel 12 is substantially vertical and the filter 14 comprises a cylindrical filter shown in cross-section in FIG. 16. The first chamber is defined within the filter and the second chamber is defined external to the filter and bounded by one or more walls, for example a cylindrical wall 228. The vessel comprises a lower region 17, provided below the filter 14 and tapering towards the lower port 22, for example having an inverted frustoconical shape and a wall 204, with the lower port 22 provided at or adjacent to the apex of the first region 17.

Referring to FIGS. 15 and 17, a variant of embodiment 220 comprises a vessel comprising first 14a and second 14b planar filters, divided into a first chamber 18 and second chamber portions 16a, 16b in fluid communication with the first chamber via filters 14a and 14b respectively. The second chamber portions 16a, 16b are connected to each other either within the device or by a fluidic pathway provided as part of an external fluidic system. In an exemplary construction the filters 14a, 14b are mounted between body components 234, 246, 238 as shown in FIG. 17, the first chamber and second chamber portions being defined by recesses and apertures formed within the body components, the body components than being assembled together. The lower region 17 tapers towards the lower port 22 and comprises either a frustoconical portion or a pyramidal shape as described before.

A method of use of the device 220 with the fluidic system as in FIG. 15 follows the steps as set out for embodiment 200. Here the second chamber may be primed and vented through breather 226, optionally sealable by valve 224 during the step of unloading suspension into syringe 44, and gas source 210 is optionally connected to upper port 82 by means of a 3 way valve 230, openable to a breather 232 that allows venting and pressure equalisation during unloading. In this fluidic system, fluid pathway 36 for unloading does not pass through valve means, so avoiding risk of losses of suspension, while inlets from the source of suspension 40 and source of wash liquid 42 are controlled by cut-off valves 38a, 38b.

In some embodiments the suspension comprises cells and the liquid comprises an undesirable component that is to be washed out. An example is in the field of cell therapy where cells to be administered to a patient may be provided in a liquid containing cryoprotectant or culture medium components unsuitable for administration to the patient, which need to be washed out, the cells then being resuspended in an excipient liquid suitable for administration, for example saline or buffer. Additionally the cells may need to be concentrated relative to the original suspension. The filter device of the invention and the fluidic system and its components may have characteristics selected from the following.

| | |
|---|---|
| Volume of the first chamber | 0.5-250 ml |
| Volume of the second chamber | 0.5-250 ml - e.g. the second chamber |
| | may be larger than the first if it |
| | comprises the waste reservoir 48 |
| Area of the filter | 1-300 cm² |
| Length of the first chamber | 2-50 cm |
| Length of the second chamber | Substantially the same as the length |
| | of the first |
| Type of filter | A variety of commercially available |
| | filters may be used according to |
| | the species to be separated. A |
| | hydrophilic planar filter membrane, |
| | compatible with cells and (if present) |
| | cyroprotectant, such as DMSO, pore |
| | size typically 0.45-0.65 um, may be |
| | used. |
| Gas used in volume reduction | Sterile or filtered air, CO₂/air or |
| | O₂/N₂/CO₂ mixtures. |
| Material of the device | Cell compatible polymer, for example |
| | PMMA, PEEK, COC. |

The filter device of the embodiments herein may be fabricated by a range of methods standard in the art of fluidics. The device may comprise a vessel 12 formed as part of a body component, in which the vessel has thin walls in which at least in part the outer profile follows the inner profile, and may be produced by injection moulding. Supporting and strengthening features may be provided as known in the art of moulding. In some embodiments the filter device comprises a body component with the vessel, chamber and fluidic pathways fabricated within it, for example by moulding, machining or embossing in which the outer profile is independent of the inner. One or more fluidic pathways may be formed within the body of the device, for example by moulding, by bonding two sub components together to define a channel, or post moulding machining to form a channel such as an aperture through solid material. In some embodiments junctions between fluidic pathways may be formed within the body component, so as to integrate fluidic pathways with the vessel. The body component may further comprise one or more reservoirs, for example for wash liquid, a second fluid for volume reduction, or waste. In this way the filter device may be provided as part of an integrated module comprising fluidic pathways connected to the filter device and to one or more reservoirs, in some embodiment via valve means external to the module, and in some embodiments via valve means mounted on the module, the module acting as fluidic manifold.

In a further embodiment of the invention, an apparatus comprises a filter device and a fluidic system connected to the filter device comprising a control means adapted to control movements of fluids into and out from the device. An embodiment of such an apparatus is shown diagrammatically in FIG. 11. In some embodiments the control means is adapted to monitor regarding the apparatus or a process carried on by the apparatus by reading and recording data from one or more sensors forming part of the apparatus.

In a further embodiment of the invention, a system comprises an apparatus comprising a control means and a data source in data communication with the control means, the data comprising instructions to determine the movement of fluids in the apparatus. In some embodiments the data source comprises a computer and a database external to the apparatus. In some embodiments the data source comprises a data store physically associated with a reservoir housing the liquid suspension. In one embodiment the system is adapted for processing a liquid comprising cells comprising apparatus as described herein and a source of liquid comprising cells in fluid communication with a port leading into the first chamber. In one embodiment the data store is physically associated with the reservoir holding the cells.

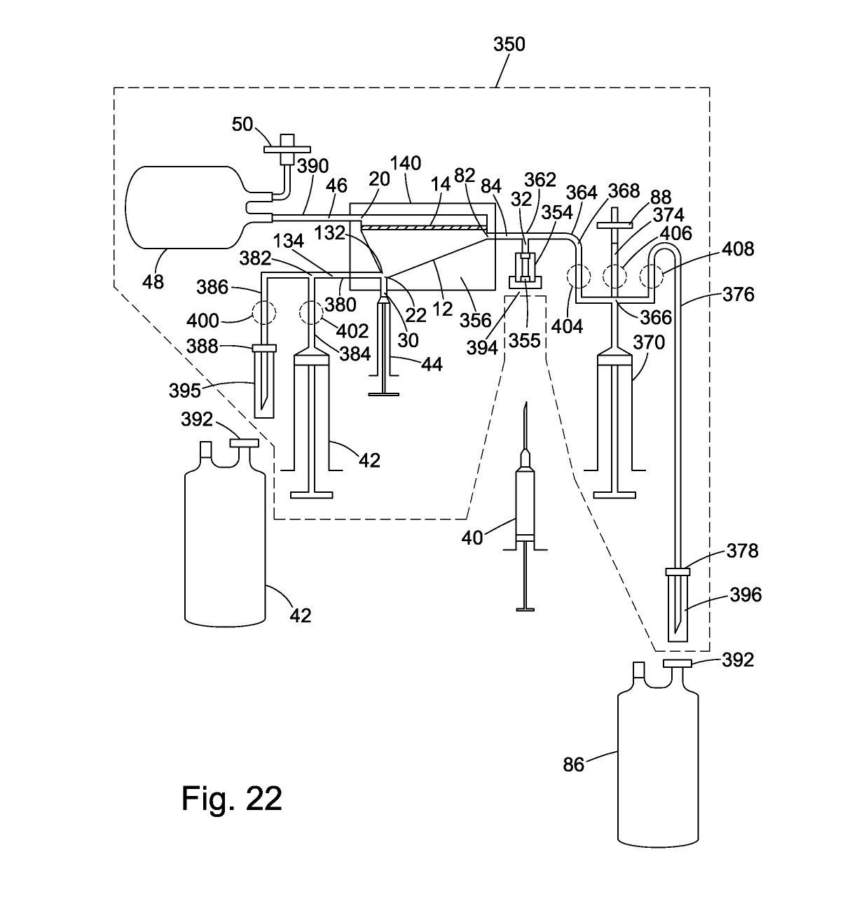

Referring to FIG. 22, an apparatus comprising a filter device as described herein is adapted to form a closed system for processing a suspension and comprises a fluidic unit 350 and a processor configured to carry out a process within the fluidic unit, the fluidic unit being adapted to interfit with the processor, the processor comprising one or more actuators configured to control fluid flow within the fluidic unit. Such an embodiment may be adapted for aseptic processing, for example for processing a cell suspension in bioprocessing, such as in processing a cell suspension for cell therapy. In this embodiment the invention provides a fluidic unit 350 comprising a filter device as described herein, the fluidic unit being configured for use with reservoirs for wash liquid 42, 86 and a source 40 of suspension to be processed, here shown in the form of a syringe having a needle. In some embodiments one or more fluidic reservoirs form part of the fluidic unit 350 and may be connected to the rest of the unit during manufacture. In FIG. 22 the reservoirs and source of suspension are shown disconnected from the fluidic unit 350 but it will be understood that when the apparatus is ready for use they are connected.

In this embodiment the fluidic unit 350 comprises:

A filter device 140 having a lower port 22 and an upper port 82 opening to the first chamber,

an inlet 354 adapted to receive a suspension,
a fluidic pathway 32 connecting the inlet to the upper port 82,
a fluidic pathway 134 configured to introduce a second liquid to the first chamber via a port 132,
an outlet fluidic pathway 30 leading from the lower port 22, and
a collection device 44 detachably connected to the outlet fluidic pathway 30.

The fluidic unit further comprises a waste reservoir 48 connected to the second chamber and optionally a breather 50 to vent gas from the waste reservoir. If the reservoir 48 is an initially empty bag then in some embodiments breather 50 is omitted.

Once the reservoirs 42 and 86 are connected the fluidic unit 350 is configured to provide a closed system.

In this embodiment the fluidic unit 350 comprises a filter device 140 comprising a vessel 12 formed within a body component 356, connected to further components of the fluidic system by means of flexible tubing. The fluidic system comprises the following tubing portions:

a tubing portion 364 connecting port 82 via T junction 362 to inlet 354 and to 4-way junction 366, and providing fluidic pathway 84 and fluidic pathway 368 connecting gas and liquid supply syringe 370 to port 82—fluidic pathway 32 connecting the inlet 354 and fluidic pathway 84 is preferably short to minimise dead space;
a tubing portion 374 connecting 4 way junction 366 to breather 88;
a tubing portion 376 connecting 4 way junction 366 to liquid connector 378;
a tubing portion 380 connecting port 132 to T junction 382;
a tubing portion 384 connecting T junction 382 to liquid supply syringe 42;
a tubing portion 386 connecting T junction 382 to liquid connector 388, and
a tubing portion 390 connecting the outlet port 20 from the second chamber of the filter device to the waste reservoir 48.

The tubing portions, junctions and other components such as the connectors may be joined by means known in the art, for example tube welding or using connection means such as Luer connectors. Inlet 354 and connectors 378 and 388 are preferably adapted to allow aseptic connection to the syringe 40 and the liquid reservoirs 42 and 86. For example the inlet 354 comprises for example a septum 355 under a sterile cap 394, and the connectors 378, 388 comprise sterile spike connections 395, 396 adapted to interfit with sterile inlet septa 392 on reservoirs in the form of bags, as known in the art. In further embodiments having substantially the same components the inlet 354 is adapted for use with a supply of suspension in a vial having a septum lid in place of syringe 40, the inlet comprising a sterile spike adapted to pierce the vial septum in place of the inlet device comprising a septum shown in FIG. 22. In further embodiments also the suspension may be introduced into the first chamber by means of aspiration at the filtrate fluid pathway 46 as described for previous embodiments.

In some embodiments the fluidic unit 350 comprises a housing adapted to retain the components of the fluidic unit such that the fluidic unit may be mounted on the processor so that actuators provided on the processor to come into contact with one or more portions of the tubing and/or one or more reservoirs. Referring to FIG. 22, as the fluidic unit 350 is mounted on the processor tubing portions 386, 384, 364, 374 and 376 come into contact with valve actuators 400, 402, 404, 406 and 408 respectively, provided on the processor, to form a pinch valve in each of the said tubing portions and syringes 42, 44 and 370 engage with syringe actuators provided on the processor.

In some embodiments the fluidic unit may comprise one or more of a temperature sensor, a heater, and electrical means to connect a sensor or a heater to a processor. This provision allows the temperature within the fluidic unit to be controlled by the processor on which the unit is mounted.

A method of use of an apparatus comprising a fluidic unit 350 and a processor comprises the following steps:

(i) the fluidic unit 350 is mounted on a processor such the one or more actuators provided on the processor engage with one or more portions of the fluidic unit,
(ii) a source of suspension is connected to the inlet 354,
(iii) the control means provided as part of the processor is initiated to follow a program to carry out a filtration process within the fluidic unit under the control of the control means, and
(iv) processed suspension is unloaded from the filter device to a collection device 44.

In some embodiments the method further comprises the step of disconnecting the collection device 44 from the outlet of the filter device.

Steps in a method of operation of an apparatus comprising the fluidic unit 350 may comprise the following:

(i) fluidic unit 350 is mounted on the processor
(ii) reservoirs 42 and 86 are connected to connectors 378 and 388
(iii) syringes 42 and 370 are filled by liquid drawn in from reservoirs 42 and 86 via pinch valves 400, 402 and 408, and the filter device is primed from one or both syringes
(iv) syringe 40 is connected to the inlet 354 and suspension is introduced into the first chamber
(v) wash liquid is flowed from syringes 42 and 370 via pinch valves 402, 404 to wash the suspension, with filtrate flowing to the waste reservoir 48
(vi) syringe 370 is emptied of liquid and filled with air via breather 88 and pinch valve 406
(vii) air is introduced into the first chamber via pinch valve 404 to concentrate the suspension
(viii) concentrated suspension is withdrawn into syringe 44
(ix) syringe 44 is detached from the fluidic system and removed from the fluidic unit.

In this way in some embodiments the filter device and apparatus are adapted to achieve processing of a cell suspension, for example for use in cell therapy, in which a component is at least partially removed from the cell suspension by the washing process, and may be replaced by the wash liquid. The wash liquid may be an excipient, and thereby the suspension is processed ready for administration to a patient. In some embodiments the fluidic unit and the processor form a closed system for processing such a cell therapy suspension.

It will be understood that a fluidic unit may comprise a filter device according to the invention and alternative fluidic components and pathways to those described above.

Preferred features of the second and subsequent aspects of the invention are as for the first aspect mutatis mutandis.

The invention will now be further described by way of reference to the following Examples which are present for the purposes of illustration only and are not to be construed as being limitations on the invention.

### Experimental Devices and Apparatus

Filter devices and apparatus of the invention were adapted for diafiltration (or washing) of a cell suspension to remove cryoprotectant from the suspension and to replace it with PBS (phosphate buffered saline), and then to concentrate the washed suspension and to load it into a collection device (a syringe).

Examples of filter devices according to the invention are shown in FIGS. 19 and 20, the two devices having the same features but being of different geometry. The example filter device is substantially as shown diagrammatically in FIGS. 9a and 9b, and the same features have the same numerals. In each case the filter device 300 comprises a body component 301 comprising a lower body part 302 and an upper body part 304 adapted to fit together so as to define the vessel 12. The body parts are adapted to be bolted together via holes, optionally tapped (not shown). The lower body part has an upper surface 306 here provided on an upstand 310, adapted to fit together with a lower surface 308 on the upper body part provided in a recess 312. The first chamber 18 is formed as a recess in the upper surface 306, having a perimeter 318 on the surface, and the second chamber 16 is formed as a recess in the lower surface 308, having a perimeter 320 on the surface. The body parts are adapted to retain a filter membrane (not shown) between them and each to form a liquid tight seal to the filter membrane outside the perimeters 318, 320 when the body components are held together.

The first and second chambers are in the form of a longer cone and a shorter cone joined at their bases, split across the diameter of the cones. The filter area between the chambers is substantially a kite shape as shown in FIG. 9b, the filter membrane being sized to extend beyond the perimeters of the first and second chambers. The sample and second chambers have substantially the same geometry being mirror-images in the plane of the surfaces when joined, which is also the plane of the membrane. The ports 22, 82 and outlet port 20 are provided as openings to the wall of the vessel, and the lower wall 142 of the first chamber slopes at an angle to the plane of the membrane, denoted by 26 in FIG. 9a, and here referred to in the figure captions as alpha. The first or lower port 22 opens to the wall of the first chamber at the join of the two cones, that is at the lowest point of the first chamber as shown in FIG. 9a. The first port is provided as part of a threaded recess 316 adapted to receive a male thread Luer connector via which a collection device such as a syringe may be connected, optionally via a 2-way valve means to control the opening or closing of the fluidic pathway 30 from the first port. Fluidic pathways 84 to port 82 and 46 from port 20 are formed as channels within the body parts.

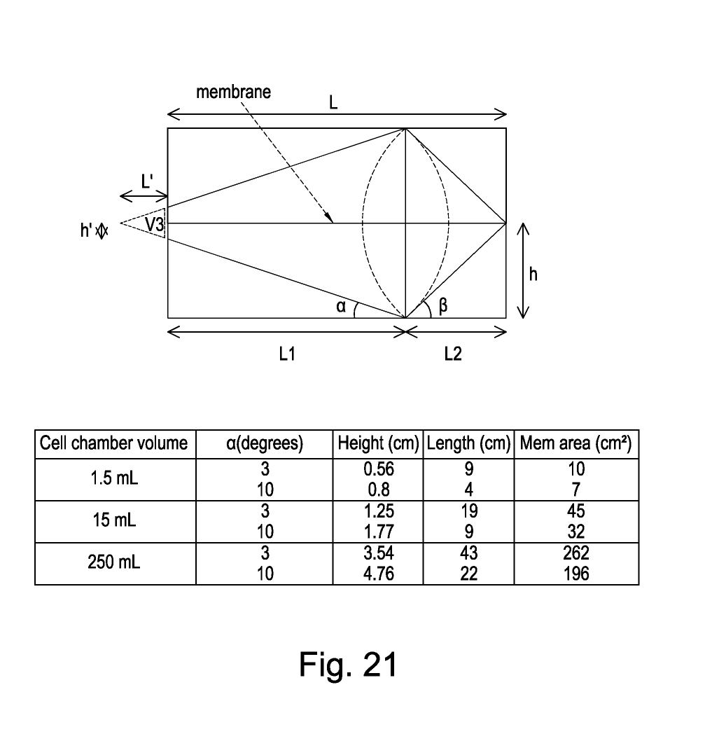

The geometry and dimensions of the example filter devices are shown in the captions to FIGS. 19 and 20, as illustrated in FIG. 21. Alpha (degrees) is the angle of the longer cone, which is also the angle of slope of the lower wall 142 to the plane of the filter, i.e. also the angle numbered as 26 in FIG. 9a of the lower wall 142 to horizontal if the device is oriented with the filter horizontal. L (cm) is the total length of the two cones and hence of the long axis of the filter, h (cm) is the height of the first chamber perpendicular from the plane of the surface 306 to the circumference of the base of the cones, which is this example lies within port 22, and A (cm2) is the total area of the filter membrane between the two chambers defined by perimeters 318 and 320. The devices shown in FIGS. 19 and 20 both have a volume in the first chamber below the plane of surface 306 of 1.5 ml. As is shown, for a smaller angle of slope alpha the device is longer and shallower to achieve the same first chamber volume.

Referring to FIG. 21, the device as shown in FIGS. 19 and 20 may be scaled up while maintaining the same angle alpha to provide a larger first chamber volume. The invention is not limited to a specific set of dimensions, shape of the vessel or first or second chambers, or angle alpha.

The example devices were machined in two parts 302, 304 as shown from cast PMMA (acrylic) and ports were formed by drilling into the wall of the half vessels. Polymer tubing was inserted into the drilled holes to form the fluidic pathways to the ports. A filter membrane was mounted between the lower and the upper halves of the filter device body using an O-ring set into the lower body part (not shown) and the two body parts were bolted together. Standard Luer fittings were used for the side inlet port (82) and the filtrate outlet port (20). The lower inlet/outlet port (22) was drilled and tapped to receive a cut-off valve (syringe valve SLLV, SGE analytical science) having a male Luer fitting.

Filter membranes used in the successful experiments included regenerated cellulose (RC) with 0.45 μm pores (Regenerated Cellulose, Type 184, 0.45 μm, Sartorius GmbH) and PVDF with 0.65 μm pores (DURAPORE hydrophilic PVDF, Millipore). Filter materials were used as supplied without pre-treatment and trimmed to shape with scissors.

For experiments the device was mounted with the filter substantially horizontal and a fluidic system as in FIG. 11 was set up, with the following differences: suspension was loaded into the first chamber from a vial replacing syringe 40 by means of aspiration by a syringe connected to port 20; the tap 38 was a 2-way syringe valve and wash liquid syringe 42 was manually connected to the lower outlet of the syringe valve in place of the product syringe 44 during the wash step.

### Experimental Protocol

Devices were primed with phosphate buffered saline (PBS).

Loading of suspension. 1-3×10⁷ Human foetal lung fibroblast cells type MRC-5 suspended in 1 ml of CryoStor 5 (BioLife) were introduced into the first chamber via the side port (82) at 2 ml/min via negative pressure at the outlet port 20. The cell suspension was denser than the buffer and was seen to flow down the sloping lower wall 142 of the first chamber to form a dense layer in the base of the chamber.

Washing. Cells were washed with PBS either (i) from the bottom up via port 22, (ii) from the side via port 82, or (iii) both simultaneously, each for 10 min. Flow rate at the lower port 22 was either 2 ml/min or 5 ml/min and from the side port 82 either zero or 5 ml/min, so the maximum volume of wash buffer passed through the suspension was 70 ml. Samples of the filtrate were taken every 30 s to 1 min for later DMSO analysis by HPLC.

Volume reduction. Air was introduced under positive pressure from syringe 152 via port 82 at a flow rate of 1 ml/min. Introduction of air was stopped when the meniscus appeared to have reached the end of the first chamber.

Unloading of concentrated suspension. Any residual pressure in the first chamber was vented by opening side port 82 to atmosphere. Concentrated cell suspension was unloaded via port 22 and the syringe valve into a pre-weighed syringe 44.

Cell counting. The syringe was weighed to find total mass of sample, cells were live/dead stained using trypan blue and counted in the concentrate and in the initial sample of thawed cells to find recovery rates for total cells and for viable cells.

Centrifugation control: cell samples as above were diluted 10× with PBS and centrifuged at 300 g for 3 min at room temperature. Supernatant was removed and the pellet was resuspended in 300 μl PBS. Cells were stained using trypan blue, counted and compared to the initial count to find the recovery rates for total cells and viable cells.

### Example 1

Results for DMSO washout were as follows:
flow from the upper port 82 only at 5 ml/min: 34%
flow from lower port 22 at 0.5 ml/min plus from upper port 82 at 5 ml/min: 62%
flow from lower port 22 at 2 ml/min plus from upper port 82 at 5 ml/min: 83%
flow from lower port 22 only at 2 ml/min: 86%.

The results show efficient washing out of DMSO (83-86% washout) for flow upwards from the bottom port 22 at 2.0 ml/min, with less efficient washing for slower flow from the base or flow from the upper port only. Washout from the centrifuge control was measured to be 80% in line with that expected from the volume ratio used in resuspension.

### Example 2

Volume reduction by introduction of air into the first chamber.

In seven repeat runs the volume recovered after volume reduction from a 1.5 ml sample was found to have a mean of 300 μl and a standard deviation of 100 μl.

### Example 3

Mean total cells recovered following washing with flow for 10 mins were as follows:
3-4 ml/min flow from the lower port only (n=3): 52%
2 ml/min flow from the lower port only (n=6): 62%
2 ml/min from the lower port and 5 ml/min from the upper port (n=6): 71%
centrifugation control (n=6): 77%.

The results show that higher flow rates from the lower port only result in lower recovery, and addition of flow from the upper port appears to increase recovery over flow from the lower port alone at the same lower port flow rate. Without being held to theory or a mechanism it is proposed that the flow from the upper port sideways over the surface of the filter membrane acts to reduce the tendency of cells to adhere to the filter, which may result from a recirculating flow pattern that acts to keep cells at a distance from the filter. Such recirculating flow patterns were observed during the tests in this example.

Recovery rate of viable cells as a proportion of viable cells in the initial suspension in the same experiment were as follows:
3-4 ml/min flow from the lower port only: 53%
2 ml/min flow from the lower port only: 62%
2 ml/min flow from the lower port and 5 ml/min from the upper port: 82%
centrifugation control: 76%.

Viable cell recovery is higher with flow from both the upper port and the lower port compared with flow from the lower port alone and similar to the recovery rate of viable cells in the centrifugation control, showing the benefit of the flow regime of upward flow from the base and sideways flow across the membrane surface as provided by the device and apparatus of the invention.

In these examples both the total and the viable cell recovery rates are not significantly different from the centrifugation control, showing that the filter device and apparatus is capable of DMSO washout, volume reduction and cell recovery performance comparable with manual centrifugation, therefore providing an efficient and automatable means for washing and concentrating cell suspensions.

### Example 4

In this example cells were used that had been cultured according to the same protocol as used for Examples 1 to 3, except that instead of a standard Styrofoam container, a cooling device, CoolCell SV10 (Biocision, Inc, San Rafael, Calif., USA, used according to the manufacturer's instructions), was used to control the rate of cooling of the cell suspension from 4 C to −80° C. Comparison tests using the centrifugation protocol showed that this resulted in higher cell viability following thawing, as measured by trypan blue exclusion tests on the as-thawed cells and on cells plated after thawing followed by 24 hours in culture. Additionally, it was observed that the cell recovery after centrifugation is directly proportional to the 24 hour cell plating efficiency of the post thaw (and pre-centrifugation) cell suspension. This evidence suggests that the most of the cell recovery variability is explained by a variable quality of the input cell suspension.

Samples (n=4) of cell suspension were thawed and sample volumes in the range 0.82 to 1.09 ml in volume were loaded into the filter device as in the preceding examples. The samples were washed with flow of wash buffer from the lower port 22 only at 2 ml/min for 20 minutes, followed by volume reduction in which air was introduced into the first chamber until it was judged by eye to occupy substantially the whole of the area of the filter. Concentrated cells were then unloaded into a syringe as before. Results in this example were:
Viability of the concentrated cells was greater than 90%,
Recovery of viable cells as a proportion of viable cells in the sample was 84+/−1%
Volume reduction was 2.7+/−0.5 fold, with output concentrate volumes in the range 320 to 420 μl.
Centrifugation control experiments (5 min at 300 g, resuspended in 300 μl PBS) on the same cell samples resulted in recovery of 91+/−9%.

These results show that flow of wash buffer from the lower port only can also result in good recovery of viable cells, similar to the results from centrifugation control experiments. Without wishing to be bound by theory, the results suggest that the efficiency of cell recovery in this filtration device is dependent on the quality of the input cells, just as in the centrifugation controls. The higher quality of this particular cell batch is the most likely cause for the average cell recovery to be higher (and the standard deviation lower) with flow from port 22 only, when compared with Example 3 results.

Thus, it will be understood that the properties of cells in such thawed suspensions may vary between cell types, cell culture and freeze/thaw protocols and the device and method provide embodiments that may selected to process suspensions with such a range of properties. In particular, in some embodiments flow only from a lower port may be used for part of the washing process and flow from an upper port might be introduced for part of the washing process, for example to move cells away from the upper region of the first chamber, so distancing them from the filter.

The invention provides devices and methods for processing suspensions, such as cell suspensions, for example in processing thawed cell suspensions to remove cryoprotectant, that provide results comparable with present manual centrifugation protocols, but which are automatable and may be carried out as part of a closed system. In this way the devices and methods enable such processing to be carried out in situations in which the manual, open system processing methods of the prior art are unsuitable or disadvantageous, such as processing of cell therapy products at the point of care.

1. A device for filtration of a liquid containing a species in suspension comprising a vessel, the vessel comprising a filter that divides the vessel into a first chamber and a second chamber, the first chamber comprising a first region and a second region wherein the second region is above the first region, the vessel being provided with:
(i) a lower port opening to the first region of the first chamber, configured to permit exit of a liquid containing the species from the first chamber,
(ii) an upper port opening to the second region of the first chamber, and
(iii) an outlet port opening to the second chamber.

2. A device according to claim 1 wherein a lower port is configured to receive a liquid containing the species.

3. A device according to claim 1 wherein an upper port is configured to receive a liquid containing the species.

4. Apparatus for filtration of a liquid containing a species in suspension comprising:
A filter device comprising a vessel, the vessel comprising a filter that divides the vessel into a first chamber and a second chamber, the first chamber comprising a first region distal to a first end of the filter and a second region proximal to the said first end of the filter, the vessel being provided with:
(i) a first port opening to the first region of the chamber, the first port being configured to receive a liquid containing the species and further configured to permit exit of the liquid containing the species from the first chamber, and
(ii) an outlet port opening to the second chamber,
and
a fluidic system in fluid communication with the filter device comprising:
(i) a collection device configured to receive a liquid containing the species from the first port
(ii) a first reservoir configured to contain a second fluid and a first fluidic pathway from the first reservoir to the first chamber,
(iii) a second reservoir configured to retain liquid from the filter device and a second fluidic pathway from the second chamber to the second reservoir, and
(iv) a fluidic actuation means provided in one of the first and the second fluidic pathways.

5. Apparatus according to claim 4 comprising a second port opening to the first chamber.

6. An apparatus according to claim 4 or 5 wherein the second region of the first chamber is above the first region.

7. A filter device or an apparatus according to any preceding claim comprising two or more ports opening to the first region of the first chamber.

8. A filter device or apparatus according any preceding claim wherein a port opens at or adjacent to the lowest point in the surface of the first chamber.

9. A filter device or apparatus according to any preceding claim wherein the first region tapers towards a port

10. A filter device or apparatus according to any preceding claim wherein a port is an opening in the surface of the first chamber.

11. A filter device or apparatus according to any preceding claim wherein a port is an opening to a conduit extending through a surface of the first chamber.

12. A filter device or apparatus according to any preceding claim wherein the first chamber has triangular cross-section.

13. A filter device or apparatus according to any preceding claim wherein the first chamber has a circular cross-section.

14. A filter device or apparatus according to any preceding claim wherein the first chamber comprises a lower wall and a planar upper wall spaced apart from the lower wall, wherein the filter is a planar filter forming part of the upper wall.

15. A filter device or apparatus according to any preceding claim wherein at least a region of a wall of the first chamber is inclined at a first angle to the plane of the filter.

16. A filter device or apparatus according to claim 15 wherein the first angle is between approximately 3 and approximately 30 degrees.

17. A filter device or apparatus according to claim 15 wherein the first angle is between approximately 30 and approximately 85 degrees.

18. A filter device or apparatus according to any preceding claim comprising a second port opening to the first chamber wherein the first chamber tapers towards the second port.

19. Apparatus for filtration of a liquid containing cells in suspension comprising a filter device as claimed in any preceding claim.

20. Apparatus comprising a filter device according to any preceding claim comprising a collection device connected to the lower port, the apparatus being configured to allow flow of a suspension from the lower port to the collection device.

21. Apparatus comprising a filter device according to any preceding claim and a reservoir containing a second fluid in fluid communication with the first chamber.

22. Apparatus according to claim 21 wherein the second fluid is a liquid.

23. Apparatus according to claim 21 wherein the second fluid is a gas.

24. Apparatus according to any claim above wherein the filter is inclined at a second angle to the horizontal.

25. Apparatus according to claim 24 wherein the second angle is in the range approximately 45 to approximately 90 degrees.

26. Apparatus according to any claim above further comprising a control means configured to control movement of fluids into and out from the filter device.

27. A system according to claim 26 further comprising a data source in data communication with the control means, the data comprising instructions to determine the movement of fluids in the apparatus.

28. A system according to claim 27 wherein the data source comprises a computer and a database external to the apparatus.

29. A system according to claim 27 wherein the data source comprises a data store physically associated with a reservoir housing the liquid suspension to be introduced to the filter device.

30. A system for processing a liquid comprising cells comprising apparatus according to any preceding claim and a source of liquid comprising cells in fluid communication with a port leading into the first chamber.

31. A method for filtration of a liquid containing a species in suspension using a filter device or apparatus according to any preceding claim, the method comprising the steps of:
(i) introducing the liquid to the first chamber,
(ii) introducing a second fluid into the first chamber
(iii) unloading the liquid containing the species from the first chamber through the first port.

32. A method according to claim 31 wherein the second fluid is a second liquid.

33. A method according to claim 32 wherein the second liquid is introduced through a port opening to the first region of the first chamber.

34. A method according to claim 33 wherein the second liquid is introduced through both of a port opening to the first region of the first chamber and an port opening to the second region.

35. A method according claim 31 wherein the second fluid is a gas.

36. A method according to claim 35 wherein gas is introduced through the upper port opening to the upper region of the first chamber.

37. A method according to claim 36 or claim 36 wherein the gas is introduced into the first chamber until the volume of liquid within the first chamber is reduced by between approximately 50% and approximately 98%.

38. A method according to claim 37 wherein the gas is introduced into the first chamber until the volume of liquid within the first chamber is reduced by between approximately 60% and approximately 95%.

39. A method according to any of claims 35 to 38 wherein the gas is introduced into the first chamber by application at the lower port or upper port of a pressure greater than atmospheric pressure.

40. A method according to any of claims 35 to 38 wherein the gas is introduced into the first chamber by application of a pressure at the outlet port of the second chamber less than atmospheric pressure.

41. A method according to claim 31 comprising the steps of:
(i) introducing a suspension into the first chamber.
(ii) preventing flow through the outlet fluidic pathway from the first chamber.
(iii) introducing a wash liquid into the first chamber.
(iv) introducing a gas into the first chamber to displace liquid through the filter and effect volume reduction in the first chamber.
(v) permitting flow through the outlet fluidic pathway from the first port and unloading the liquid containing the species from the first chamber.

42. A method according to claim 41 wherein steps (iii) and (iv) are repeated in order to achieve diafiltration by means of sequential volume reduction and dilution.

43. A method according to any preceding claim further comprising the step of allowing a species in the sample to sediment within the first chamber.

44. A method according to any preceding claim further comprising a backflush step of introducing liquid from the second chamber into the first chamber through the filter.

45. A method according to any preceding claim further comprising the step of tilting the device during the filtration process from a first position at which the lower wall of the first chamber is at a first angle to horizontal to a second position at which the lower wall of the first chamber is at a second angle to horizontal.

46. A method according to any preceding claim wherein the device is vibrated during at least a portion of the filtration process.

47. A method according to any preceding claim including a step wherein the control means reads instructions from a data source so as to control the flow of at least one fluid into or out of the filter device.

48. A method according to any preceding claim wherein the liquid is a cell suspension comprising a cryoprotectant.

49. A method as claimed in claim 48 further comprising a step of resuspending the cell suspension in an excipient.

50. A cell suspension obtained by a method according to any of claims 31 to 49.
